(19) United States
(12) Patent Application Publication
(10) Pub. No.: US 2022/0010327 A1
(43) Pub. Date: Jan. 13, 2022

(54) INSECTICIDAL PROTEINS

(71) Applicant: Syngenta Participations AG, Basel (CH)

(72) Inventor: Clarence Michael Reynolds, Research Triangle Park, NC (US)

(73) Assignee: Syngenta Participations AG, Basel (CH)

(21) Appl. No.: 17/289,335

(22) PCT Filed: Oct. 18, 2019
(86) PCT No.: PCT/US2019/056982

§ 371 Date: Apr. 28, 2021

Related U.S. Application Data
(60) Provisional application No. 62/752,500, filed on Oct. 30, 2018.

(51) Int. Cl. C12N 15/82; C07K 14/195

(52) U.S. Cl. CPC C12N 15/8286; C07K 14/195

(57) ABSTRACT

Compositions and methods for controlling plant pests are disclosed. In particular, novel chimeric insecticidal proteins having toxicity to at least coleopteran insect pests are provided. Nucleic acid molecules encoding the novel insecticidal proteins are also provided. Methods of making the insecticidal proteins and methods of using the insecticidal proteins and nucleic acids encoding the insecticidal proteins of the invention, for example in transgenic plants to confer protection from insect damage, are also disclosed.

### FIELD OF THE INVENTION

The present invention relates to the fields of protein engineering, plant molecular biology and pest control. More particularly the invention relates to chimeric proteins having insecticidal activity, nucleic acids whose expression results in the chimeric insecticidal proteins, and methods of making and methods of using the chimeric insecticidal proteins and corresponding nucleic acids to control insects.

### BACKGROUND

Insect pests are a major cause of crop losses. In the United States alone, billions of dollars are lost every year due to infestation by various genera of insects. In addition to losses in field crops, insect pests are also a burden to vegetable and fruit growers to producers of ornamental flowers, and they are a nuisance to gardeners and homeowners.

Species of corn rootworm are considered to be the most destructive corn pests. In the United States alone, three species, Diabrotica virgifera virgifera, the western corn rootworm, D. longicornis barberi, the northern corn rootworm and D. undecimpunctata howardi, the southern corn rootworm, cause over one billion dollars in damage to corn each year in the US corn belt. An important corn rootworm pest in the Southern US is the Mexican corn rootworm, Diabrotica virgifera zeae. In South America, Diabrotica speciosa is considered to be an important pest of corn. Western corn rootworm spread to Europe in 1992 and since 2008 has been causing economic damage throughout the major corn growing regions. Corn rootworm larvae cause the most substantial plant damage by feeding almost exclusively on corn roots. This injury has been shown to increase plant lodging, to reduce grain yield and vegetative yield as well as alter the nutrient content of the grain. Larval feeding also causes indirect effects on corn by opening avenues through the roots for bacterial and fungal infections which lead to root and stalk rot diseases. Adult corn rootworms are active in cornfields in late summer where they feed on ears, silks and pollen, thus interfering with normal pollination.

Corn rootworms are mainly controlled by intensive applications of chemical pesticides, which are active through inhibition of insect growth, prevention of insect feeding or reproduction, or cause death. Good corn rootworm control can thus be reached, but these chemicals can sometimes also affect other, beneficial organisms. Another problem resulting from the wide use of chemical pesticides is the appearance of resistant insect varieties. Yet another problem is due to the fact that corn rootworm larvae feed underground thus making it difficult to apply rescue treatments of insecticides. Therefore, most insecticide applications are made prophylactically at the time of planting. This practice results in a large environmental burden. This has been partially alleviated by various farm management practices, but there is an increasing need for alternative pest control mechanisms.

Biological pest control agents, such as Bacillus thuringiensis (Bt) strains expressing pesticidal toxins like δ-endotoxins (delta-endotoxins; also called crystal toxins or Cry proteins), have been applied to crop plants with satisfactory results against insect pests. The δ-endotoxins are proteins held within a crystalline matrix that are known to possess insecticidal activity when ingested by certain insects. Several native Cry proteins from Bacillus thuringiensis, or engineered Cry proteins, have been expressed in transgenic crop plants and exploited commercially to control certain lepidopteran and coleopteran insect pests. For example, starting in 2003, transgenic corn hybrids that control corn rootworm by expressing a Cry3Bb1, Cry34Ab1/Cry35Ab1 or modified Cry3A (mCry3A) or eCry3.1Ab protein have been available commercially in the US.

Although the use of transgenic plants expressing Cry proteins has been shown to be extremely effective, insect pests that now have resistance against the Cry proteins expressed in certain transgenic plants are known. Therefore, there remains a need to identity new and effective pest control agents that provide an economic benefit to farmers and that are environmentally acceptable. Particularly needed are proteins that are toxic to Diabrotica species, a major pest of corn, that have a different mode of action than existing insect control products as a way to mitigate the development of resistance. Furthermore, delivery of insect control agents through products that minimize the burden on the environment, as through transgenic plants, are desirable.

### SUMMARY

In view of these needs, the present invention provides novel chimeric insecticidal proteins constructed using domains from proteins isolated from bacteria in the genus Serratia and related bacteria. The insecticidal proteins from which the domains of the chimeric proteins are derived are SproCRW (SEQ ID NO:1), SplyCRW (SEQ ID NO:2), SquiCRW (SEQ ID NO:3), Plu1415 (SEQ OID NO:17) or WoodsCRW (SEQ ID NO:4), their variants, and proteins which are substantially identical to SproCRW, SplyCRW, SquiCRW, Plu1415 or WoodsCRW and their variants. The Chimeric insecticidal proteins of the invention have toxicity to at least coleopteran insect pests, particularly to corn rootworm (Diabrotica spp) pests. The invention is further drawn to nucleic acid molecules that encode a chimeric insecticidal protein and/or a variant thereof, their complements, or which are substantially identical to a chimeric insecticidal protein and/or a variant thereof.

Also provided by the invention are vectors containing such recombinant (or complementary thereto) nucleic acids; a plant or microorganism which includes and enables expression of such nucleic acids; plants transformed with such nucleic acids, for example transgenic corn plants; the progeny of such plants which contain the nucleic acids stably incorporated and hereditable in a Mendelian manner, and/or the seeds of such plants and such progeny. The invention also includes methods of breeding to introduce a transgene comprising a nucleic acid molecule of the invention into a progeny plant and into various germplasms.

The invention also provides compositions and formulations containing the chimeric insecticidal proteins of the invention or variants thereof, which are capable of inhibiting the ability of insect pests to survive, grow and/or reproduce, or of limiting insect-related damage or loss to crop plants, for example applying a chimeric insecticidal protein, or variants thereof, as part of compositions or formulations to insect-infested areas or plants, or to prophylactically treat insect-susceptible areas or plants to confer protection against the insect pests.

The invention is further drawn to a method of making a chimeric insecticidal protein of the invention, or variants thereof, and to methods of using the nucleic acids, for example in microorganisms to control insects or in transgenic plants to confer protection from insect damage.

The novel chimeric insecticidal proteins described herein are active against insects. For example, in some embodiments, the proteins of the invention can be used to control economically important insect pests, including coleopteran insects such as western corn rootworm (WCR; Diabrotica virgifera virgifera), northern corn rootworm (NCR; D. longicornis barberi), southern corn rootworm (SCR; D. undecimpunctata howardi) and/or Mexican corn rootworm (MCR; D. virgifera zeae). The chimeric insecticidal proteins of the invention can be used singly or in combination with other insect control strategies to confer enhanced pest control efficiency against the same insect pest and/or to increase the spectrum of target insects with minimal environmental impact.

Other aspects and advantages of the present invention will become apparent to those skilled in the art from a study of the following description of the invention and non-limiting examples.

### BRIEF DESCRIPTION OF THE SEQUENCES IN THE SEQUENCE LISTING

SEQ ID NO: 1 is a Serratia proteamaculans SproCRW amino acid sequence.
SEQ ID NO: 2 is a Serratia plymuthica SplyCRW amino acid sequence.
SEQ ID NO: 3 is a Serratia quinivorans SquiCRW amino acid sequence.
SEQ ID NO:4 is a WoodsCRW amino acid sequence.
SEQ ID NO:5 is a SplyCRW/SproCRW amino acid sequence.
SEQ ID NO:6 is a SproCRW/SplyCRW amino acid sequence.
SEQ ID NO:7 is a SproCRW/WoodsCRW amino acid sequence.
SEQ ID NO:8 is a WoodsCRW/SproCRW amino acid sequence.
SEQ ID NO:9 is a SplyCRW/WoodsCRW amino acid sequence.
SEQ ID NO:10 is a WoodsCRW/SplyCRW amino acid sequence.
SEQ ID NO:11 is a SplyCRW/SproCRW E. coli optimized nucleotide sequence.
SEQ ID NO:12 is a SproCRW/SplyCRW E. coli optimized nucleotide sequence.
SEQ ID NO:13 is a SproCRW/WoodsCRW E. coli optimized nucleotide sequence.
SEQ ID NO:14 is a WoodsCRW/SproCRW E. coli optimized nucleotide sequence.
SEQ ID NO:15 is a SplyCRW/WoodsCRW E. coli optimized nucleotide sequence.
SEQ ID NO:16 is a WoodsCRW/SplyCRW E. coli optimized nucleotide sequence.
SEQ ID NO:17 is a Plu1415 amino acid sequence.
SEQ ID NO:18 is a Plu1415/SproCRW chimeric amino acid sequence.
SEQ ID NO:19 is a Plu1415/SplyCRW chimeric amino acid sequence.
SEQ ID NO:20 is a Plu1415/SproCRW chimeric E. coli optimized nucleotide sequence.
SEQ ID NO:21 is a Plu1415/SplyCRW chimeric E. coli optimized nucleotide sequence.
SEQ ID NO:22 is a Plu1415/HmassCRW chimeric amino acid sequence.
SEQ ID NO:23 is a Plu1415/Hmass chimeric E. coli optimized nucleotide sequence.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

For clarity, certain terms used in the specification are defined and presented as follows:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the word “and/or” refers to and encompasses any and all possible combinations of one or more of the associated listed items, as well as the lack of combinations when interpreted in the alternative, “or.”

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

“Activity” of the insecticidal proteins of the invention is meant that the insecticidal proteins function as orally active insect control agents, have a toxic effect, and/or are able to disrupt or deter insect feeding, which may or may not cause death of the insect. When an insecticidal protein of the invention is delivered to the insect, the result is typically death of the insect, or the insect does not feed upon the source that makes the insecticidal protein available to the insect. “Pesticidal” is defined as a toxic biological activity capable of controlling a pest, such as an insect, nematode, fungus, bacteria, or virus, preferably by killing or destroying them. “Insecticidal” is defined as a toxic biological activity capable of controlling insects, preferably by killing them. A “pesticidal agent” is an agent that has pesticidal activity. An “insecticidal agent” is an agent that has insecticidal activity.

The term “chimeric construct” or “chimeric gene” or “chimeric polynucleotide” or “chimeric nucleic acid” or “chimeric protein” (or similar terms) as used herein refers to a construct or nucleic acid molecule or protein comprising two or more polynucleotides or amino acid motifs or domains, respectively, of different origin assembled into a single nucleic acid molecule or protein. The term “chimeric construct”, “chimeric gene”, “chimeric polynucleotide” or “chimeric nucleic acid” refers to any construct or molecule that contains, without limitation, (1) polynucleotides (e.g., DNA), including regulatory and coding polynucleotides that are not found together in nature (i.e., at least one of the polynucleotides in the construct is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In some embodiments of the invention, the chimeric construct, chimeric gene, chimeric polynucleotide or chimeric nucleic acid comprises an expression cassette comprising a polynucleotide of the invention under the control of regulatory polynucleotides, particularly under the control of regulatory polynucleotides functional in plants or bacteria.

The term “chimeric protein” refers to a protein that comprises, consists essentially of or consists of amino acid sequences, for example motifs or domains that from two or more different sources. Such sources may be domains or motifs from different insecticidal proteins that when combined into one chimeric protein, make a non-naturally occurring chimeric insecticidal protein.

A “coding sequence” (also called CDS) is a nucleic acid sequence that is transcribed into RNA such as mRNA, rRNA, tRNA, snRNA, sense RNA or antisense RNA. Preferably the RNA is then translated in an organism to produce a protein.

To “control” insects means to inhibit, through a toxic effect, the ability of insect pests to survive, grow, feed, and/or reproduce, or to limit insect-related damage or loss in crop plants. To “control” insects may or may not mean killing the insects, although it preferably means killing the insects.

As used herein, a “codon optimized” sequence means a nucleotide sequence wherein the codons are chosen to reflect the particular codon bias that a host cell or organism may have. This is typically done in such a way so as to preserve the amino acid sequence of the polypeptide encoded by the nucleotide sequence to be optimized. In certain embodiments, the DNA sequence of the recombinant DNA construct includes sequence that has been codon optimized for the cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, incorporated herein by reference.

To “control” insects means to inhibit, through a toxic effect, the ability of insect pests to survive, grow, feed, or reproduce, or to limit insect-related damage or loss in crop plants or to protect the yield potential of a crop when grown in the presence of insect pests. To “control” insects may or may not mean killing the insects, although it preferably means killing the insects.

The terms “comprises” or “comprising,” when used in this specification, specify the presence of stated features, integers, steps, operations, elements, or components, but do not preclude the presence or addition of one or more other features, integers, steps, operations, elements, components, or groups thereof.

As used herein, the transitional phrase “consisting essentially of” (and grammatical variants) means that the scope of a claim is to be interpreted to encompass the specified materials or steps recited in the claim “and those that do not materially alter the basic and novel characteristic(s)” of the claimed invention. Thus, the term “consisting essentially of” when used in a claim of this invention is not intended to be interpreted to be equivalent to “comprising.”

In the context of the invention, “corresponding to” or “corresponds to” means that when the amino acid sequences of insecticidal proteins or variant or homologs thereof are aligned with each other, the amino acids that “correspond to” certain enumerated positions in the variant or homolog protein are those that align with these positions in a reference protein but that are not necessarily in these exact numerical positions relative to the particular reference amino acid sequence of the invention. For example, if SEQ ID NO:1 is the reference sequence and is aligned with SEQ ID NO:2, amino acid Asn at position 420 (Asn420) of SEQ ID NO:2 “corresponds to” an Asn at position 421 (Asn421) of SEQ ID NO:1, or for example, Asn424 of SEQ ID NO:2 “corresponds to” Gly425 of SEQ ID NO:1.

To “deliver” a composition or toxic protein means that the composition or toxic protein comes in contact with an insect, which facilitates the oral ingestion of the composition or toxic protein, resulting in a toxic effect and control of the insect. The composition or toxic protein can be delivered in many recognized ways, including but not limited to, transgenic plant expression, formulated protein composition(s), sprayable protein composition(s), a bait matrix, or any other art-recognized protein delivery system.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide group.

“Effective insect-controlling amount” means that concentration of an insecticidal protein that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may have at least one of its components heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

An expression cassette comprising a nucleotide sequence of interest may be chimeric, meaning that at least one of its components is heterologous with respect to at least one of its other components. An expression cassette may also be one that comprises a native promoter driving its native gene, however it has been obtained in a recombinant form useful for heterologous expression. Such usage of an expression cassette makes it so it is not naturally occurring in the cell into which it has been introduced.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used. Any available terminator known to function in plants can be used in the context of this invention.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

“Gene of interest” refers to any nucleic acid molecule which, when transferred to a plant, confers upon the plant a desired trait such as antibiotic resistance, virus resistance, insect resistance, disease resistance, or resistance to other pests, herbicide tolerance, abiotic stress tolerance, male sterility, modified fatty acid metabolism, modified carbohydrate metabolism, improved nutritional value, improved performance in an industrial process or altered reproductive capability. The “gene of interest” may also be one that is transferred to plants for the production of commercially valuable enzymes or metabolites in the plant.

A “heterologous” nucleic acid sequence or nucleic acid molecule is a nucleic acid sequence or nucleic acid molecule not naturally associated with a host cell into which it is introduced, including non-naturally occurring multiple copies of a naturally occurring nucleic acid sequence. A heterologous nucleic acid sequence or nucleic acid molecule may comprise a chimeric sequence such as a chimeric expression cassette, where the promoter and the coding region are derived from multiple source organisms. The promoter sequence may be a constitutive promoter sequence, a tissue-specific promoter sequence, a chemically-inducible promoter sequence, a wound-inducible promoter sequence, a stress-inducible promoter sequence, or a developmental stage-specific promoter sequence.

A “homologous” nucleic acid sequence is a nucleic acid sequence naturally associated with a host cell into which it is introduced.

“Homologous recombination” is the reciprocal exchange of nucleic acid fragments between homologous nucleic acid molecules.

The term “identity” or “identical” or “substantially identical,” in the context of two nucleic acid or amino acid sequences, refers to two or more sequences or subsequences that have at least 60%, preferably at least 80%, more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues or bases in length, more preferably over a region of at least about 100 residues or bases, and most preferably the sequences are substantially identical over at least about 150 residues or bases. In an especially preferred embodiment, the sequences are substantially identical over the entire length of the coding regions. Furthermore, substantially identical nucleic acid or amino acid sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (National Center for Biotechnology Information, U.S. National Library of Medicine, 8600 Rockville Pike, Bethesda, Md. 20894 USA). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but not to other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

The term “isolated” nucleic acid molecule, polynucleotide or protein is a nucleic acid molecule, polynucleotide or protein that no longer exists in its natural environment. An isolated nucleic acid molecule, polynucleotide or protein of the invention may exist in a purified form or may exist in a recombinant host such as in a transgenic bacteria or a transgenic plant. Therefore, a claim to an “isolated” nucleic acid molecule, polynucleotide or protein, as enumerated herein, encompasses a nucleic acid molecule, polynucleotide or protein when the nucleic acid molecule or polynucleotide is comprised within a transgenic plant genome or the protein is expressed in the transgenic plant.

A “nucleic acid molecule” or “nucleic acid sequence” or “polynucleotide” is a segment of single- or double-stranded DNA or RNA that can be isolated from any source. In the context of the present invention, the nucleic acid molecule, nucleic acid sequence or polynucleotide is typically a segment of DNA. In some embodiments, the nucleic acid molecule, nucleic sequence or polynucleotide of the invention are isolated.

“Operably linked” refers to the association of polynucleotides on a single nucleic acid fragment so that the function of one affects the function of the other. For example, a promoter is operably linked with a coding polynucleotide or functional RNA when it is capable of affecting the expression of that coding polynucleotide or functional RNA (i.e., that the coding polynucleotide or functional RNA is under the transcriptional control of the promoter). Coding polynucleotide in sense or antisense orientation can be operably linked to regulatory polynucleotides.

As used herein “pesticidal,” insecticidal,” and the like, refer to the ability of a chimeric protein of the invention to control a pest organism or an amount of a chimeric protein that can control a pest organism as defined herein. Thus, a pesticidal chimeric protein can kill or inhibit the ability of a pest organism (e.g., insect pest) to survive, grow, feed, or reproduce.

The terms “protein,” “peptide” and “polypeptide” are used interchangeably herein.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A “polynucleotide” refers to a polymer composed of many nucleotide monomers covalently bonded in a chain. Such “polynucleotides” includes DNA, RNA, modified oligo nucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

“Polynucleotide of interest” refers to any polynucleotide which, when transferred to an organism, e.g., a plant, confers upon the organism a desired characteristic such as insect resistance, disease resistance, herbicide tolerance, antibiotic resistance, improved nutritional value, improved performance in an industrial process, production of commercially valuable enzymes or metabolites or altered reproductive capability.

A “promoter” is an untranslated DNA sequence upstream of the coding region that contains the binding site for RNA polymerase and initiates transcription of the DNA. The promoter region may also include other elements that act as regulators of gene expression.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule incorporated into its genome. As a result of such genomic alteration, the recombinant plant is distinctly different from the related wild-type plant.

“Regulatory elements” refer to sequences involved in controlling the expression of a nucleotide sequence. Regulatory elements comprise a promoter operably linked to the nucleotide sequence of interest and termination signals. They also typically encompass sequences required for proper translation of the nucleotide sequence.

“Transformation” is a process for introducing heterologous nucleic acid into a host cell or organism. In particular embodiments, “transformation” means the stable integration of a DNA molecule into the genome (nuclear or plastid) of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

This invention provides compositions and methods for controlling harmful plant pests. Particularly, the invention relates to novel chimeric insecticidal proteins which have activity against at least coleopteran insects, for example, Diabrotica virgifera virgifera (western corn rootworm; WCR), Diabrotica barberi (northern corn rootworm; NCR), and/or Diabrotica undecimpunctata howardi (southern corn rootworm; SCR) and/or other Diabrotica species including Diabrotica virgifera zeae (Mexican corn rootworm), and/or Leptinotarsa decimlineata (Colorado potato beetle; CPB). In some embodiments, a novel chimeric insecticidal protein of the invention may have activity against other insect pests, including lepidopteran insect pests, including without limitation Agrotis ipsilon (black cutworm), Diatraea saccharalis (sugar cane borer; SCB) and/or Diatraea grandiosella (southwestern corn borer; SWCB). The present invention also relates to nucleic acids whose expression results in chimeric insecticidal proteins of the invention, and to the making and using of the chimeric insecticidal proteins to control insect pests. In embodiments, the expression of the nucleic acids results in chimeric insecticidal proteins that can be used to control at least coleopteran insect pests such as western corn rootworm, northern corn rootworm and/or southern corn rootworm, particularly when expressed in a transgenic plant such as a transgenic corn plant.

The invention further encompasses a nucleic acid molecule comprising a nucleotide sequence that encodes a chimeric insecticidal protein of the invention. The nucleotide sequence may be optimized for expression in bacteria, such as Escherichia coli, or for expression in a plant, such as corn (Zea mays). A nucleotide sequence optimized for expression in a heterologous organism, such as a species of bacteria or plant different from where the sequence originated, is not naturally occurring. In one aspect of this embodiment, the nucleic acid molecule comprises, consists essentially of or consists of a nucleotide sequence of any of SEQ ID NOs:11-16, 20, 21 and/or 23. Specifically exemplified teachings of methods to make nucleic acid molecules that encode the chimeric insecticidal proteins of the invention can be found in the examples of the present application. Those skilled in the art will recognize that modifications can be made to the exemplified methods to make the chimeric insecticidal proteins encompassed by the present invention.

A skilled person would recognize that a transgene for commercial use, such as a nucleic acid molecule that comprises any of SEQ ID NO:11-16, 20, 21 and/or 23, may have relatively minor modifications to the nucleic acid sequence to comply with governmental regulatory standards. Such modifications would not affect the function of the resulting, molecule, which would be substantially identical to SEQ ID NO:11-16, 20, 21 and/or 23. A skilled person would recognize that the modified nucleic acid molecule would be essentially the same as the starting molecule, and is encompassed by the present invention.

In some embodiments, the invention encompasses a chimeric insecticidal protein that is toxic to an insect pest, comprising, consisting essentially of or consisting of in an N-terminal to C-terminal direction (a) an N-terminal region comprising, consisting essentially of or consisting of an amino acid sequence that corresponds to amino acid 1 to amino acid 338, 339, 340, 341, 342, 343, 344, 345, 346, 347, 348, 349, 350, 351 or 352 of (i) SEQ ID NO:1 or an amino acid sequence that has at least 80% identity to SEQ ID NO:1; or (ii) SEQ ID NO:2 or an amino acid sequence that has at least 80% identity to SEQ ID NO:2; or (iii) SEQ ID NO:3 or an amino acid sequence that has at least 80% identity to SEQ ID NO:3; or (iv) SEQ ID NO:4 or an amino acid sequence that has at least 80% identity to SEQ ID NO:4, fused to (b) a C-terminal region comprising, consisting essentially of or consisting of an amino acid sequence that corresponds to amino acid 339, 340, 341, 342, 343, 344, 345, 346, 347, 348, 349, 350, 351, 352 or 353 to amino acid 488, 489 or 490 of (i) SEQ ID NO:1, or an amino acid sequence that has at least 80% identity to SEQ ID NO:1; or (ii) SEQ ID NO:2 or an amino acid sequence that has at least 80% identity to SEQ ID NO:2; or (iii) SEQ ID NO:3 or an amino acid sequence that has at least 80% identity to SEQ ID NO:3.

In some embodiments, the N-terminal region of a chimeric insecticidal protein of the invention comprises, consists essentially of or consists of (a) an amino acid sequence that corresponds to amino acid 1 to amino acid 345 of SEQ ID NO:1 and the C-terminal region comprises an amino acid sequence that corresponds to amino acid 346 to amino acid 488 of SEQ ID NO:2; or (b) an amino acid sequence that corresponds to amino acid 1 to amino acid 345 of SEQ ID NO:2 and the C-terminal region comprises, consists essentially of or consists of an amino acid sequence that corresponds to amino acid 346 to amino acid 489 of SEQ ID NO:1; or (c) an amino acid sequence that corresponds to amino acid 1 to amino acid 346 of SEQ ID NO:4 and the C-terminal region comprises, consists essentially of or consists of an amino acid sequence that corresponds to amino acid 346 to amino acid 489 of SEQ ID NO:1; or (d) an amino acid sequence that corresponds to amino acid 1 to amino acid 346 of SEQ ID NO:4 and the C-terminal region comprises, consists essentially of or consists of an amino acid sequence that corresponds to amino acid 346 to amino acid 488 of SEQ ID NO:2.

In other embodiments, the N-terminal region of a chimeric insecticidal protein of the invention comprises, consists essentially of or consists of (a) amino acid 1 to amino acid 345 of SEQ ID NO:1 and the C-terminal region comprises, consists essentially of or consists of amino acid 346 to amino acid 488 of SEQ ID NO:2; or (b) amino acid 1 to amino acid 345 of SEQ ID NO:2 and the C-terminal region comprises, consists essentially of or consists of amino acid 346 to amino acid 489 of SEQ ID NO:1; or (c) amino acid 1 to amino acid 346 of SEQ ID NO:4 and the C-terminal region comprises, consists essentially of or consists of amino acid 346 to amino acid 489 of SEQ ID NO:1; or (d) amino acid 1 to amino acid 346 of SEQ ID NO:4 and the C-terminal region comprises, consists essentially of or consists of amino acid 346 to amino acid 488 of SEQ ID NO:2.

In other embodiments, a chimeric insecticidal protein of the invention comprises, consists essentially of or consists of (a) an amino acid sequence that has at least 80% identity to SEQ ID NO:5, SEQ ID NO:6, SEQ ID NO:8 or SEQ ID NO:10; or (b) an amino acid sequence of SEQ ID NO:5, SEQ ID NO:6, SEQ ID NO:8 or SEQ ID NO:10.

In some embodiments, the invention encompasses a chimeric insecticidal protein that is toxic to an insect pest, comprising, consisting essentially of or consisting of in an N-terminal to C-terminal direction (a) an N-terminal region comprising, consisting essentially of or consisting of an amino acid sequence that corresponds to amino acid 1 to about amino acid 363 of SEQ ID NO:17, or an amino acid sequence that has at least 80% identity to SEQ ID NO:17, fused to (b) a C-terminal region comprising, consisting essentially of or consisting of (i) an amino acid sequence that corresponds to about amino acid position 347 to about amino acid position 489 of SEQ ID NO:1, or an amino acid sequence that has at least 80% identity to SEQ ID NO:1; or (ii) an amino acid sequence that corresponds to about amino acid position 346 to about amino acid position 488 of SEQ ID NO:2, or an amino acid sequence that has at least 80% identity to SEQ ID NO:2.

In some embodiments, the N-terminal region of a chimeric insecticidal protein of the invention comprises, consists essentially of or consists of (a) an amino acid sequence that corresponds to amino acid 1 to amino acid 363 of SEQ ID NO:17 and the C-terminal region comprises an amino acid sequence that corresponds to amino acid 347 to amino acid 489 of SEQ ID NO:1; or (b) an amino acid sequence that corresponds to amino acid 1 to amino acid 363 of SEQ ID NO:17 and the C-terminal region comprises, consists essentially of or consists of an amino acid sequence that corresponds to amino acid 346 to amino acid 488 of SEQ ID NO:2.

In other embodiments, a chimeric insecticidal protein of the invention comprises, consists essentially of or consists of (a) an amino acid sequence that has from at least 80% identity to at least 99% identity to SEQ ID NO:18 or SEQ ID NO:19; or (b) an amino acid sequence of SEQ ID NO:18 or SEQ ID NO:19.

The chimeric insecticidal proteins of the invention have insect control activity when tested against insect pests in bioassays or when expressed in transgenic organisms that are feed upon by insect pests. In some embodiments, the chimeric insecticidal proteins of the invention are active against at least coleopteran insect pests. Insects in the order Coleoptera include but are not limited to any coleopteran insect now known or later identified including those in suborders Archostemata, Myxophaga, Adephaga and Polyphaga, and any combination thereof.

In other embodiments, the chimeric insecticidal proteins of the invention are active against Diabrotica species. Diabrotica is a genus of beetles of the order Coleoptera commonly referred to as “corn rootworms” or “cucumber beetles.” Exemplary Diabrotica species include without limitation Diabrotica barberi (northern corn rootworm), D. virgifera virgifera (western corn rootworm), D. undecimpunctata howardii (southern corn rootworm), D. balteata (banded cucumber beetle), D. undecimpunctata undecimpunctata (western spotted cucumber beetle), D. significata (3-spotted leaf beetle), D. speciosa (chrysanthemum beetle), D. virgifera zeae (Mexican corn rootworm), D. beniensis, D. cristata, D. curviplustalata, D. dissimilis, D. elegantula, D. emorsitans, D. graminea, D. hispanloe, D. lemniscata, D. linsleyi, D. milleri, D. nummularis, D. occlusal, D. porrecea, D. scutellata, D. tibialis, D. trifasciata and D. viridula; and any combination thereof.

Other nonlimiting examples of Coleopteran insect pests according to the present invention include Leptinotarsa spp. such as L. decemlineata (Colorado potato beetle); Chrysomela spp. such as C. scripta (cottonwood leaf beetle); Hypothenemus spp. such as H. hampei (coffee berry borer); Sitophilus spp. such as S. zeamais (maize weevil); Epitrix spp. such as E. hirtipennis (tobacco flea beetle) and E. cucumeris (potato flea beetle); Phyllotreta spp. such as P. cruciferae (crucifer flea beetle) and P. pusilla (western black flea beetle); Anthonomus spp. such as A. eugenii (pepper weevil); Hemicrepidus spp. such as H. memnonius (wireworms); Melanotus spp. such as M. communis (wireworm); Ceutorhychus spp. such as C. assimilis (cabbage seedpod weevil); Phyllotreta spp. such as P. cruciferae (crucifer flea beetle); Aeolus spp. such as A. mellillus (wireworm); Aeolus spp. such as A. mancus (wheat wireworm); Horistonotus spp. such as H. uhlerii (sand wireworm); Sphenophorus spp. such as S. maidis (maize billbug), S. zeae (timothy billbug), S. parvulus (bluegrass billbug), and S. callosus (southern corn billbug); Phyllophaga spp. (White grubs); Chaetocnema spp. such as C. pulicaria (corn flea beetle); Popillia spp. such as P. japonica (Japanese beetle); Epilachna spp. such as E. varivestis (Mexican bean beetle); Cerotoma spp. such as C. trifurcate (Bean leaf beetle); Epicauta spp. such as E. pestifera and E. lemniscata (Blister beetles); and any combination of the foregoing.

Chimeric insecticidal proteins of the invention may also be active against insect pests in the order Lepidoptera. Insects in the order Lepidoptera include without limitation any insect now known or later identified that is classified as a lepidopteran, including those insect species within suborders Zeugloptera, Glossata, and Heterobathmiina, and any combination thereof. Exemplary lepidopteran insects include, but are not limited to, Ostrinia spp. such as O. nubilalis (European corn borer); Plutella spp. such as P. xylostella (diamondback moth); Spodoptera spp. such as S. frugiperda (fall armyworm), S. ornithogalli (yellowstriped armyworm), S. praefica (western yellowstriped armyworm), S. eridania (southern armyworm) and S. exigua (beet armyworm); Agrotis spp. such as A. ipsilon (black cutworm), A. segetum (common cutworm), A. gladiaria (claybacked cutworm), and A. orthogonia (pale western cutworm); Striacosta spp. such as S. albicosta (western bean cutworm); Helicoverpa spp. such as H. zea (corn earworm), H. punctigera (native budworm), S. littoralis (Egyptian cotton leafworm) and H. armigera (cotton bollworm); Heliothis spp. such as H. virescens (tobacco budworm); Diatraea spp. such as D. grandiosella (southwestern corn borer) and D. saccharalis (sugarcane borer); Trichoplusia spp. such as T. ni (cabbage looper); Sesamia spp. such as S. nonagroides (Mediterranean corn borer); Pectinophora spp. such as P. gossypiella (pink bollworm); Cochylis spp. such as C. hospes (banded sunflower moth); Manduca spp. such as M. sexta (tobacco hornworm) and M. quinquemaculata (tomato hornwonn); Elasmopalpus spp. such as E. lignosellus (lesser cornstalk borer); Pseudoplusia spp. such as P. includens (soybean looper); Anticarsia spp. such as A. gemmatalis (velvetbean caterpillar); Plathypena spp. such as P. scabra (green cloverworm); Pieris spp. such as P. brassicae (cabbage butterfly), Papaipema spp. such as P. nebris (stalk borer); Pseudaletia spp. such as P. unipuncta (common armyworm); Peridroma spp. such as P. saucia (variegated cutworm); Keiferia spp. such as K. lycopersicella (tomato pinworm); Artogeia spp. such as A. rapae (imported cabbageworm); Phthorimaea spp. such as P. operculella (potato tuberworm); Crymodes spp. such as C. devastator (glassy cutworm); Feltia spp. such as F. ducens (dingy cutworm); and any combination of the foregoing. In one aspect of this embodiment, the chimeric insecticidal proteins of the invention are active against black cutworm, sugar cane borer, and/or southwestern corn borer.

The chimeric insecticidal proteins of the invention may also be active against Hemipteran, Dipteran, Lygus spp., and/or other piercing and sucking insects, for example of the order Orthoptera or Thysanoptera. Insects in the order Diptera include but are not limited to any dipteran insect now known or later identified including but not limited to Liriomyza spp. such as L. trifolii (leafminer) and L. sativae (vegetable leafminer); Scrobipalpula spp. such as S. absoluta (tomato leafminer); Delia spp. such as D. platura (seedcorn maggot), D. brassicae (cabbage maggot) and D. radicum (cabbage root fly); Psilia spp. such as P. rosae (carrot rust fly); Tetanops spp. such as T. myopaeformis (sugarbeet root maggot); and any combination of the foregoing.

Insects in the order Orthoptera include but are not limited to any orthopteran insect now known or later identified including but not limited to Melanoplus spp. such as M. differentialis (Differential grasshopper), M. femurrubrum (Redlegged grasshopper), M. bivittatus (Twostriped grasshopper); and any combination thereof.

Insects in the order Thysanoptera include but are not limited to any thysanopteran insect now known or later identified including but not limited to Frankliniella spp. such as F. occidentalis (western flower thrips) and F. fusca (tobacco thrips); and Thrips spp. such as T. tabaci (onion thrips), T. palmi (melon thrips); and any combination of the foregoing.

The chimeric insecticidal proteins of the invention may also be active against nematodes. The term “nematode” as used herein encompasses any organism that is now known or later identified that is classified in the animal kingdom, phylum Nematoda, including without limitation nematodes within class Adenophorea (including for example, orders Enoplida, Isolaimida, Mononchida, Dorylaimida, Trichocephalida, Mennithida, Muspiceida, Araeolaimida, Chromadorida, Desmoscolecida, Desmodorida and Monhysterida) and/or class Secernentea (including, for example, orders Rhabdita, Strongylida, Ascaridida, Spirurida, Camallanida, Diplogasterida, Tylenchida and Aphelenchida).

Nematodes include but are not limited to parasitic nematodes such as root-knot nematodes, cyst nematodes and/or lesion nematodes. Exemplary genera of nematodes according to the present invention include but are not limited to, Meloidogyne (root-knot nematodes), Heterodera (cyst nematodes), Globodera (cyst nematodes), Radopholus (burrowing nematodes), Rotylenchulus (reniform nematodes), Pratylenchus (lesion nematodes), Aphelenchoides (foliar nematodes), Helicotylenchus (spiral nematodes), Hoplolaimus (lance nematodes), Paratrichodorus (stubby-root nematodes), Longidorus, Nacobbus (false root-knot nematodes), Subanguina, Belonlaimus (sting nematodes), Criconemella, Criconemoides (ring nematodes), Ditylenchus, Dolichodorus, Hemicriconemoides, Hemicycliophora, Hirschmaniella, Hypsoperine, Macroposthonia, Melinius, Punctodera, Quinisulcius, Scutellonema, Xiphinema (dagger nematodes), Tylenchorhynchus (stunt nematodes), Tylenchulus, Bursaphelenchus (round worms), and any combination thereof.

Exemplary plant parasitic nematodes according to the present invention include, but are not limited to, Belonolaimus gracilis, Belonolaimus longicaudatus, Bursaphelenchus xylophilus (pine wood nematode), Criconemoides ornata, Ditylenchus destructor (potato rot nematode), Ditylenchus dipsaci (stem and bulb nematode), Globodera pallida (potato cyst nematode), Globodera rostochiensis (golden nematode), Heterodera glycines (soybean cyst nematode), Heterodera schachtii (sugar beet cyst nematode); Heterodera zeae (corn cyst nematode), Heterodera avenae (cereal cyst nematode), Heterodera carotae, Heterodera trifolii, Hoplolaimus columbus, Hoplolaimus galeatus, Hoplolaimus magnistylus, Longidorus breviannulatus, Meloidogyne arenaria, Meloidogyne chitwoodi, Meloidogyne hapla, Meloidogyne incognita, Meloidogyne javanica, Mesocriconema xenoplax, Nacobbus aberrans, Naccobus dorsalis, Paratrichodorus christiei, Paratrichodorus minor, Pratylenchus brachyurus, Pratylenchus crenatus, Pratylenchus hexincisus, Pratylenchus neglectus, Pratylenchus penetrans, Pratylenchus projectus, Pratylenchus scribneri, Pratylenchus tenuicaudatus, Pratylenchus thornei, Pratylenchus zeae, Punctodera chaccoensis, Quinisulcius acutus, Radopholus similis, Rotylenchulus reniformis, Tylenchorhynchus dubius, Tylenchulus semipenetrans (citrus nematode), Siphinema americanum, X. Mediterraneum, and any combination of the foregoing.

The chimeric insecticidal proteins of the invention can be used in combination with other pesticidal agents (e.g. Bt Cry proteins) to increase pest target range. Furthermore, the use of the chimeric insecticidal proteins of the invention in combination with an insecticidal agent which has a different mode of action or target a different receptor in the insect gut has particular utility for the prevention and/or management of insect resistance.

The second pesticidal agent may be an insecticidal protein derived from Bacillus thuringiensis. A B. thuringiensis insecticidal protein can be any of a number of insecticidal proteins including but not limited to a Cry1 protein, a Cry3 protein, a Cry7 protein, a Cry8 protein, a Cry11 protein, a Cry22 protein, a Cry 23 protein, a Cry 36 protein, a Cry37 protein, a Cry34 protein together with a Cry35 protein, a binary chimeric insecticidal protein CryET33 and CryET34, a binary insecticidal protein TIC100 and TIC101, a binary insecticidal protein PS149B1, a Vegetative Insecticidal Proteins (VIPs), for example, those disclosed in U.S. Pat. Nos. 5,849,870 and 5,877,012, herein incorporated by reference, a TIC900 or related protein, a TIC901, TIC1201, TIC407, TIC417, a modified Cry3A protein, or hybrid proteins or chimeric proteins made from any of the preceding insecticidal proteins. In other embodiments, the B. thuringiensis insecticidal protein is selected from the group consisting of Cry3Bb1, Cry34Ab1 together with Cry35Ab1, rnCry3A (U.S. Pat. No. 7,276,583, incorporated herein by reference), eCry3.1Ab (U.S. Pat. No. 8,309,516, incorporated herein by reference), and Vip3A proteins, including Vip3Aa (U.S. Pat. No. 6,137,033, incorporated herein by reference).

In some embodiments, the invention encompasses a nucleic acid molecule comprising, consisting essentially of or consisting of (a) a nucleotide sequence encoding a chimeric insecticidal protein comprising an amino acid sequence having from at least 80% to at least 99% identity to any of SEQ ID NOs:5-10, 18, 19 or 22; or (b) a nucleotide sequence of (a) that is codon optimized for expression in a transgenic organism.

In other embodiments, a nucleic acid molecule of the invention is codon optimized for expression in a transgenic bacteria and/or a transgenic plant. In some aspects of these embodiments, the bacteria is in the genus Bacillus, Clostridium, Xenorhabdus, Photorhabdus, Pasteuria, Escherichia, Pseudomonas, Erwinia, Serratia, Klebsiella, Salmonella, Pasteurella, Xanthomonas, Streptomyces, Rhizobium, Rhodopseudomonas, Methylophilius, Agrobacterium, Acetobacter, Lactobacillus, Arthrobacter, Azotobacter, Leuconostoc, or Alcaligenes. In other aspects, a nucleic acid molecule of the invention is codon optimized for expression in a transgenic Escherichia coli bacteria. In still other aspects, the plant is a dicot plant or a monocot plant. In still other aspects the dicot plant is selected from the group consisting of a soybean, sunflower, tomato, cole crop, cotton, sugar beet and tobacco; or the monocot plant is selected from the group consisting of a barley, corn, oat, rice, sorghum, sugar cane and wheat. In further aspects, the nucleic acid molecule is codon optimized for expression in a transgenic corn plant. In still further aspects, a nucleic acid molecule of the invention that is codon optimized for expression in a transgenic organism comprises, consists essentially of or consists of a nucleotide sequence of any of SEQ ID NOs:11-16, 20, 21 or 23.

In some embodiments, the invention encompasses a chimeric gene comprising a heterologous promoter operably linked to a nucleic acid molecule of the invention. In some aspects of these embodiments, the heterologous promoter is a plant expressible promoter. In other aspects, the plant expressible promoter is selected from the group of promoters consisting of ubiquitin, cestrum yellow virus, corn TrpA, OsMADS 6, maize H3 histone, bacteriophage T3 gene 9 5′ UTR, corn sucrose synthetase 1, corn alcohol dehydrogenase 1, corn light harvesting complex, corn heat shock protein, maize mtl, pea small subunit RuBP carboxylase, rice actin, rice cyclophilin, Ti plasmid mannopine synthase, Ti plasmid nopaline synthase, petunia chalcone isomerase, bean glycine rich protein 1, potato patatin, lectin, CaMV 35S and S-E9 small subunit RuBP carboxylase promoter.

In other aspects of these embodiments, the nucleic acid molecule that encodes a chimeric insecticidal protein of the invention that is active against at least a coleopteran insect pest. In other aspects, the coleopteran insect pest is a Diabrotica insect pest. In still other aspects, the Diabrotica insect pest is selected from the group consisting of Diabrotica virgifera virgifera (western corn rootworm), Diabrotica barberi (northern corn rootworm), Diabrotica undecimpunctata howardi (southern corn rootworm) and Diabrotica zeae (Mexican corn rootworm).

The invention also encompasses recombinant vectors or recombinant constructs, which may also be referred to as vectors or constructs, comprising the expression cassettes and/or the nucleic acid molecules of the invention. In such vectors, the nucleic acids are preferably in expression cassettes comprising regulatory elements for expression of the nucleotide molecules in a host cell capable of expressing the nucleic acid molecules. Such regulatory elements usually comprise a promoter and termination signals and preferably also comprise elements allowing efficient translation of polypeptides encoded by the nucleic acids of the invention. Vectors comprising the nucleic acids may be capable of replication in particular host cells, preferably as extrachromosomal molecules, and are therefore used to amplify the nucleic acids of this invention in the host cells. The invention also encompasses a host cell that contains an expression cassette or a nucleic acid molecule of the invention. In some embodiments, host cells for such vectors are microorganisms, such as bacteria, in particular Bacillus thuringiensis or E. coli, or a fungus such as yeast. In other embodiments, host cells for such recombinant vectors are endophytes or epiphytes. In yet other embodiments, such vectors are viral vectors and are used for replication of the nucleotide sequences in particular host cells, e.g. insect cells or plant cells. Recombinant vectors are also used for transformation of the nucleic acid molecules of the invention into host cells, whereby the nucleic acid molecules are stably integrated into the DNA of a transgenic host. In some embodiments, the transgenic host is a plant, for example a monocot plant, such as corn plant. In other embodiments, the transgenic host plant is a dicot plant, such as a soybean plant or cotton plant.

In other embodiments, at least one of the nucleic acid molecules of the invention is inserted into an appropriate expression cassette, comprising a promoter and termination signal. Expression of the nucleic acid may be constitutive, or an inducible promoter responding to various types of stimuli to initiate transcription may be used. In other embodiments, the cell in which the chimeric insecticidal protein of the invention is expressed is a microorganism, such as a virus, bacteria, or a fungus. In yet other embodiments, a virus, such as a baculovirus, contains a nucleic acid molecule of the invention in its genome and expresses large amounts of the corresponding chimeric insecticidal protein after infection of appropriate eukaryotic cells that are suitable for virus replication and expression of the nucleic acid. The chimeric insecticidal protein thus produced is used as an insecticidal agent. Alternatively, baculoviruses engineered to include the nucleic acid are used to infect insects in vivo and kill them either by expression of the insecticidal toxin or by a combination of viral infection and expression of the insecticidal toxin. In a further embodiment, the invention also encompasses a method for producing a chimeric protein with insecticidal activity, comprising culturing the host cell under conditions in which the nucleic acid molecule encoding the chimeric protein of the invention is expressed.

In other embodiments, the invention also encompasses a transgenic host cell comprising a recombinant vector of the invention. In some aspects of these embodiments, the transgenic host cell is a transgenic bacterial cell or transgenic plant cell.

In other aspects, the transgenic bacterial cell is in the genus Bacillus, Clostridium, Xenorhabdus, Photorhabdus, Pasteuria, Escherichia, Pseudomonas, Erwinia, Serratia, Klebsiella, Salmonella, Pasteurella, Xanthomonas, Streptomyces, Rhizobium, Rhodopseudomonas, Methylophilius, Agrobacterium, Acetobacter, Lactobacillus, Arthrobacter, Azotobacter, Leuconostoc, or Alcaligenes. In other aspects, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera. Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the inventive nucleic acids for the same purpose. In still other aspects the transgenic bacterial cell is an Escherichia coli cell. In other aspects, the Bacillus cell is a transgenic Bacillus thuringiensis cell. Techniques for these genetic manipulations are specific for the different available hosts and are known in the art. For example, the expression vectors pKK223-3 and pKK223-2 can be used to express heterologous genes in E. coli, either in transcriptional or translational fusion, behind the tac or trc promoter. For the expression of operons encoding multiple ORFs, the simplest procedure is to insert the operon into a vector such as pKK223-3 in transcriptional fusion, allowing the cognate ribosome binding site of the heterologous genes to be used. Techniques for overexpression in gram-positive species such as Bacillus are also known in the art and can be used in the context of this invention (Quax et al. In:Industrial Microorganisms:Basic and Applied Molecular Genetics, Eds. Baltz et al., American Society for Microbiology, Washington (1993)). Alternate systems for overexpression rely for example, on yeast vectors and include the use of Pichia, Saccharomyces and Kluyveromyces (Sreekrishna, In:Industrial microorganisms:basic and applied molecular genetics, Baltz, Hegeman, and Skatrud eds., American Society for Microbiology, Washington (1993); Dequin & Barre, Biotechnology L2:173-177 (1994); van den Berg et al., Biotechnology 8:135-139 (1990)).

In still other aspects of these embodiments, the transgenic plant cell is a transgenic dicot plant cell or a transgenic monocot plant cell. In some embodiments, the transgenic dicot plant cell is selected from the group consisting of a soybean cell, sunflower cell, tomato cell, cole crop cell, cotton cell, sugar beet cell and tobacco cell; or the transgenic monocot plant cell is selected from the group consisting of a barley cell, corn cell, oat cell, rice cell, sorghum cell, sugar cane cell and wheat cell.

In some embodiments, the invention encompasses a transgenic plant or plant part comprising a nucleic acid molecule, chimeric gene, expression cassette or recombinant vector of the invention. In other embodiments, the transgenic plant or plant part expresses a chimeric insecticidal protein encoded by the nucleic acid molecule, chimeric gene, expression cassette or recombinant vector of the invention. In still other embodiments, the transgenic plant or plant part comprises any of SEQ ID NOs:5-10, 18, 19 or 22. In other embodiments, the transgenic plant or plant part is a transgenic corn plant or corn plant part.

In some embodiments, a nucleic acid molecule of the invention is expressed in transgenic plants, thus causing the biosynthesis of the corresponding chimeric insecticidal protein in the transgenic plants. In this way, transgenic plants with enhanced resistance to insects, for example corn rootworm, are generated. For their expression in transgenic plants, the nucleic acid molecules of the invention may optionally be modified and optimized. Although in many cases genes from microbial organisms can be expressed in plants at high levels without modification, low expression in transgenic plants may result from microbial nucleic acids having codons that are not preferred in plants. It is known in the art that all organisms have specific preferences for codon usage, and the codons of the nucleic acids described in this invention can be changed to conform with plant preferences, while maintaining the amino acids encoded thereby. Furthermore, high expression in plants is best achieved from coding sequences that have at least about 35% GC content, preferably more than about 45%, more preferably more than about 50%, and most preferably more than about 60%. Microbial nucleic acids that have low GC contents may express poorly in plants due to the existence of ATTTA motifs that may destabilize messages, and AATAAA motifs that may cause inappropriate polyadenylation. In some embodiments, sequences can be modified to account for the specific codon preferences and GC content preferences of monocotyledons or dicotyledons as these preferences have been shown to differ (Murray et al. Nucl. Acids Res. 17:477-498 (1989)). In addition, the nucleic acids are screened for the existence of illegitimate splice sites that may cause message truncation. All changes required to be made within the nucleic acids such as those described above can be made using well known techniques of site directed mutagenesis, PCR, and synthetic gene construction, for example, using the methods described in the published patent applications EP 0 385 962, EP 0 359 472, and WO93/07278.

In some embodiments of the invention, a coding sequence for a chimeric insecticidal protein of the invention is made according to the procedure disclosed in U.S. Pat. No. 5,625,136, herein incorporated by reference. In this procedure, maize preferred codons, i.e., the single codon that most frequently encodes that amino acid in maize, are used. The maize preferred codon for a particular amino acid might be derived, for example, from known gene sequences from maize. Maize codon usage for 28 genes from maize plants is found in Murray et al., Nucleic Acids Research 17:477-498 (1989), the disclosure of which is incorporated herein by reference.

In this manner, the nucleotide sequences can be optimized for expression in any plant. It is recognized that all or any part of the gene sequence may be optimized or synthetic. That is, synthetic or partially optimized sequences may also be used.

For more efficient initiation of translation, sequences adjacent to the initiating methionine may be modified. For example, they can be modified by the inclusion of sequences known to be effective in plants. Joshi has suggested an appropriate consensus for plants (NAR 15:6643-6653 (1987)) and Clonetech suggests a further consensus translation initiator (1993/1994 catalog, page 210). These consensus sequences are suitable for use with the nucleic acids of this invention. In embodiments, the sequences are incorporated into constructions comprising the nucleic acids, up to and including the ATG (whilst leaving the second amino acid unmodified), or alternatively up to and including the GTC subsequent to the ATG (with the possibility of modifying the second amino acid of the transgene).

Expression of nucleic acid molecules of the invention in transgenic plants is driven by promoters that function in plants. The choice of promoter will vary depending on the temporal and spatial requirements for expression, and also depending on the target species. Thus, expression of the nucleic acids of this invention in leaves, in stalks or stems, in ears, in inflorescences (e.g. spikes, panicles, cobs, etc.), in roots, and/or seedlings is preferred. In many cases, however, protection against more than one type of insect pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleic acids in the desired cell.

In some embodiments, promoters are used that are expressed constitutively including the actin or ubiquitin or cmp promoters or the CaMV 35S and 19S promoters. The nucleic acids of this invention can also be expressed under the regulation of promoters that are chemically regulated. Preferred technology for chemical induction of gene expression is detailed in the published application EP 0 332 104 (to Ciba-Geigy) and U.S. Pat. No. 5,614,395. A preferred promoter for chemical induction is the tobacco PR-1a promoter.

In other embodiments, a category of promoters which is wound inducible can be used. Numerous promoters have been described which are expressed at wound sites and also at the sites of phytopathogen infection. Ideally, such a promoter should only be active locally at the sites of infection, and in this way the chimeric insecticidal proteins of the invention only accumulate in cells that need to synthesize the proteins to kill the invading insect pest. Preferred promoters of this kind include those described by Stanford et al. Mol. Gen. Genet. 215:200-208 (1989), Xu et al. Plant Molec. Biol. 22:573-588 (1993), Logemann et al. Plant Cell 1:151-158 (1989), Rohrmeier & Lehle, Plant Molec. Biol. 22:783-792 (1993), Firek et al. Plant Molec. Biol. 22:129-142 (1993), and Warner et al. Plant J. 3:191-201 (1993).

Tissue-specific or tissue-preferential promoters useful for the expression of genes encoding chimeric insecticidal proteins of the invention in plants, particularly corn, are those which direct expression in root, pith, leaf or pollen, particularly root. Such promoters, e.g. those isolated from PEPC or trpA, are disclosed in U.S. Pat. No. 5,625,136, or MTL, disclosed in U.S. Pat. No. 5,466,785. Both U. S. patents are herein incorporated by reference in their entirety.

In addition, promoters functional in plastids can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of nucleotide sequences of the invention in a plant through the application of an exogenous chemical regulator. Regulation of the expression of nucleotide sequences of the invention via promoters that are chemically regulated enables the polypeptides of the invention to be synthesized only when the crop plants are treated with the inducing chemicals. Depending upon the objective, the promoter may be a chemical-inducible promoter, where application of a chemical induces expression of a nucleotide sequence of the invention, or a chemical-repressible promoter, where application of the chemical represses expression of a nucleotide sequence of the invention.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Intl Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In further aspects, nucleotide sequences of the invention can be operably associated with a promoter that is wound inducible or inducible by pest or pathogen infection (e.g., a insect or nematode plant pest). Numerous promoters have been described which are expressed at wound sites and/or at the sites of pest attack (e.g., insect/nematode feeding) or phytopathogen infection. Ideally, such a promoter should be active only locally at or adjacent to the sites of attack, and in this way expression of the nucleotide sequences of the invention will be focused in the cells that are being invaded or fed upon. Such promoters include, but are not limited to, those described by Stanford et al., Mol. Gen. Genet. 215:200-208 (1989), Xu et al. Plant Molec. Biol. 22:573-588 (1993), Logemann et al. Plant Cell 1:151-158 (1989), Rohrmeier and Lehle, Plant Molec. Biol. 22:783-792 (1993), Firek et al. Plant Molec. Biol. 22:129-142 (1993), Warner et al. Plant J. 3:191-201 (1993), U.S. Pat. Nos. 5,750,386, 5,955,646, 6,262,344, 6,395,963, 6,703,541, 7,078,589, 7,196,247, 7,223,901, and U.S. Patent Application Publication 2010043102.

In some embodiments of the invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

Numerous other sequences can be incorporated into expression cassettes described in this invention. These include sequences that have been shown to enhance expression such as intron sequences (e.g. from Adhl and bronzel) and viral leader sequences (e.g. from TMV, MCMV and AMV).

It may be preferable to target expression of the nucleic acids of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, the DNA encoding the target peptide from a known organelle-targeted gene product is manipulated and fused upstream of the nucleic acid. Many such target sequences are known for the chloroplast and their functioning in heterologous constructions has been shown. The expression of the nucleic acids of the present invention is also targeted to the endoplasmic reticulum or to the vacuoles of the host cells. Techniques to achieve this are well known in the art.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors comprising the nucleic acid molecules of the present invention may also comprise genes (e.g. phosphomannose isomerase; PMD which provide for positive selection of the transgenic plants as disclosed in U.S. Pat. Nos. 5,767,378 and 5,994,629, herein incorporated by reference. The choice of selectable marker is not, however, critical to the invention.

In some embodiments, the nucleic acid can be transformed into the nuclear genome. In other embodiments, a nucleic acid of the present invention is directly transformed into the plastid genome. A major advantage of plastid transformation is that plastids are generally capable of expressing bacterial genes without substantial codon optimization, and plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Nati. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleic acid of the present invention is inserted into a plastid-targeting vector and transformed into the plastid genome of a desired plant host. Plants homoplastic for plastid genomes containing a nucleic acid of the present invention are obtained, and are preferentially capable of high expression of the nucleic acid.

In some embodiments, a transgenic plant of the invention may comprise at least a second pesticidal agent which is non-proteinaceous. In some aspects of these embodiments, the second pesticidal agent is an interfering RNA molecule. An interfering RNA typically comprises at least a RNA fragment against a target gene, a spacer sequence, and a second RNA fragment which is complementary to the first, so that a double-stranded RNA structure can be formed. RNA interference (RNAi) occurs when an organism recognizes double-stranded RNA (dsRNA) molecules and hydrolyzes them. The resulting hydrolysis products are small RNA fragments of about 19-24 nucleotides in length, called small interfering RNAs (siRNAs). The siRNAs then diffuse or are carried throughout the organism, including across cellular membranes, where they hybridize to mRNAs (or other RNAs) and cause hydrolysis of the RNA. Interfering RNAs are recognized by the RNA interference silencing complex (RISC) into which an effector strand (or “guide strand”) of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes from which the mRNAs were transcribed. Interfering RNAs are known in the art to be useful for insect control (see, for example, publication WO2013/192256, incorporated by reference herein). An interfering RNA designed for use in insect control produces a non-naturally occurring double-stranded RNA, which takes advantage of the native RNAi pathways in the insect to trigger down-regulation of target genes that may lead to the cessation of feeding and/or growth and may result in the death of the insect pest. The interfering RNA molecule may confer insect resistance against the same target pest as the protein of the invention, or may target a different pest. The targeted insect plant pest may feed by chewing, sucking, or piercing. Interfering RNAs are known in the art to be useful for insect control. In other embodiments, the interfering RNA may confer resistance against a non-insect plant pest, such as a nematode pest or a virus pest.

The co-expression of more than one pesticidal agent in the same transgenic plant can be achieved by making a single recombinant vector comprising coding sequences of more than one pesticidal agent in a so called molecular stack and genetically engineering a plant to contain and express all the pesticidal agents in the transgenic plant. Such molecular stacks may be also be made by using mini-chromosomes as described, for example in U.S. Pat. No. 7,235,716. Alternatively, a transgenic plant comprising one nucleic acid encoding a first pesticidal agent can be re-transformed with a different nucleic acid encoding a second pesticidal agent and so forth. Alternatively, a plant, Parent 1, can be genetically engineered for the expression of genes of the present invention. A second plant, Parent 2, can be genetically engineered for the expression of a second pesticidal agent. By crossing Parent 1 with Parent2, progeny plants are obtained which express all the genes introduced into Parents 1 and 2.

Transgenic plants or seed comprising a chimeric insecticidal protein of the invention can also be treated with an insecticide or insecticidal seed coating as described in U.S. Pat. Nos. 5,849,320 and 5,876,739, herein incorporated by reference. Where both the insecticide or insecticidal seed coating and the transgenic plant or seed of the invention are active against the same target insect, for example a Coleopteran pest or a Diabrotica target pest, the combination is useful (i) in a method for further enhancing activity of the composition of the invention against the target insect, and (ii) in a method for preventing development of resistance to the composition of the invention by providing yet another mechanism of action against the target insect. Thus, the invention provides a method of enhancing control of a Diabrotica insect population comprising providing a transgenic plant or seed of the invention and applying to the plant or the seed an insecticide or insecticidal seed coating to a transgenic plant or seed of the invention.

Even where the insecticidal seed coating is active against a different insect, the insecticidal seed coating is useful to expand the range of insect control, for example by adding an insecticidal seed coating that has activity against lepidopteran insects to a transgenic seed of the invention, which, in some embodiments, has activity against coleopteran and some lepidopteran insects, the coated transgenic seed produced controls both lepidopteran and coleopteran insect pests.

Examples of such insecticides and/or insecticidal seed coatings include, without limitation, a carbamate, a pyrethroid, an organophosphate, a friprole, a neonicotinoid, an organochloride, a nereistoxin, or a combination thereof. In another embodiment, the insecticide or insecticidal seed coating are selected from the group consisting of carbofuran, carbaryl, methomyl, bifenthrin, tefluthrin, permethrin, cyfluthrin, lambda-cyhalothrin, cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, and a combination thereof. Commercial products containing such insecticides and insecticidal seed coatings include, without limitation, Furadan® (carbofuran), Lanate® (methomyl, metomil, mesomile), Sevin® (carbaryl), Talstar® (bifenthrin), Force® (tefluthrin), Ammo® (cypermethrin), Cymbush® (cypermethrin), Delta Gold® (deltamethrin), Karate® (lambda-cyhalothrin), Ambush® (permethrin), Pounce® (permethrin), Brigade® (bifenthrin), Capture® (bifenthrin), ProShield® (tefluthrin), Warrior® (lambda-cyhalothrin), Dursban® (chlorphyrifos), Fortress® (chlorethoxyfos), Mocap® (ethoprop), Thimet® (phorate), AAstar® (phorate, flucythinate), Rampart® (phorate), Counter® (terbufos), Cygon® (dimethoate), Dicapthon, Regent® (fipronil), Cruiser® (thiamethoxam), Gaucho® (imidacloprid), Prescribe® (imidacloprid), Poncho® (clothianidin) and Aztec® (cyfluthrin, tebupirimphos).

The invention also encompasses an insecticidal composition comprising an effective insect-controlling amount of a chimeric insecticidal protein of the invention. In further embodiments, the insecticidal composition comprises a suitable agricultural carrier and a chimeric insecticidal protein of the invention. The agricultural carrier may include adjuvants, mixers, enhancers, etc. beneficial for application of an active ingredient, such as a chimeric insecticidal protein of the invention, including a chimeric insecticidal protein comprising, consisting essentially of or consisting of an amino acid sequence that is at least 80%, at least 85%, at least 90%, at least 95%, or 100% identical to any of SEQ ID NOs:5-10, 18, 19 and/or 22. Suitable carriers should not be phytotoxic to valuable crops, particularly at the concentrations employed in applying the compositions in the presence of crops, and should not react chemically with the compounds of the active ingredient herein, namely a polypeptide of the invention, or other composition ingredients. Such mixtures can be designed for application directly to crops, or can be concentrates or formulations which are normally diluted with additional carriers and adjuvants before application. They may include inert or active components and can be solids, such as, for example, dusts, powders, granules, water dispersible granules, or wettable powders, or liquids, such as, for example, emulsifiable concentrates, solutions, emulsions or suspensions. Suitable agricultural carriers may include liquid carriers, for example water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, and the like. Water is generally the carrier of choice for the dilution of concentrates. Suitable solid carriers may include talc, pyrophyllite clay, silica, attapulgus clay, kieselguhr, chalk, diatomaceous earth, lime, calcium carbonate, bentonire clay, Fuller's earth, cotton seed hulls, wheat flour, soybean flour, pumice, wood flour, walnut shell flour, lignin, and the like. In another embodiment, a polypeptide of the invention may be encapsulated in a synthetic matrix such as a polymer and applied to the surface of a host such as a plant. Ingestion of the host cells by an insect permits delivery of the insect control agents to the insect and results in a toxic effect in the insect pest.

In further embodiments, an insecticidal composition of the invention may be a powder, dust, pellet, granule, spray, emulsion, colloid, or solution. An insecticidal composition of the invention may be prepared by desiccation, lyophilization, homogenization, extraction, filtration, centrifugation, sedimentation, or concentration of a culture of bacterial cells, for example Bacillus thuringiensis cells. A composition of the invention may comprise at least 1%, about 5%, at least 10%, at least 20%, at least 25%, at least 30%, at least 35%, at least 40%, at least 50%, at least 60%, at least 70%, at least 80%, at least 90%, at least 95%, at least 97%, or at least 99% by weight a chimeric insecticidal protein of the invention. An insecticidal composition of the invention may comprise at least a second pesticidal agent, which may be insecticidal, nematicidal, fungicidal, or bactericidal. At least a second pesticidal agent may be insecticidal to the same insect as a chimeric insecticidal protein of the invention or to a different insect. The second pesticidal agent may be a protein. The pesticidal agent may be an interfering RNA. The second pesticidal agent may be a microorganism, such as a bacteria, which comprises a nucleic acid molecule that encodes for a pesticidal agent and/or contains a pesticidal agent such as a protein or interfering RNA. The microorganism may be attenuated, heat-inactivated, or lyophilized. The microorganism may be dead or unable to reproduce. The second pesticidal agent may be an insecticide, for example arbofuran, carbaryl, methomyl, bifenthrin, tefluthrin, permethrin, cyfluthrin, lambda-cyhalothrin, cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, or a combination thereof, or a commercial product containing such insecticides and insecticidal seed coatings as described above.

An insecticidal composition of the invention, for example a composition comprising a chimeric insecticidal protein of the invention and an agriculturally acceptable carrier, may be used in conventional agricultural methods. An agriculturally acceptable carrier is a formulation useful for applying a composition comprising a polypeptide of the invention to a plant or seed. For example, the compositions of the invention may be mixed with water and/or fertilizers and may be applied preemergence and/or postemergence to a desired locus by any means, such as airplane spray tanks, irrigation equipment, direct injection spray equipment, knapsack spray tanks, cattle dipping vats, farm equipment used in ground spraying (e.g., boom sprayers, hand sprayers), and the like. The desired locus may be soil, plants, and the like.

An insecticidal composition of the invention may be applied to a seed or plant propagule in any physiological state, at any time between harvest of the seed and sowing of the seed; during or after sowing; and/or after sprouting. It is preferred that the seed or plant propagule be in a sufficiently durable state that it incurs no or minimal damage, including physical damage or biological damage, during the treatment process. A formulation may be applied to the seeds or plant propagules using conventional coating techniques and machines, such as fluidized bed techniques, the roller mill method, rotostatic seed treaters, and drum coaters.

In some embodiments, the invention encompasses a method for producing a chimeric insecticidal protein of the invention, comprising culturing a host cell of the invention or an organism comprising the host cell under conditions in which the host cell produces the chimeric insecticidal protein.

In some embodiments, the invention also encompasses a method of producing a transgenic plant or plant part having enhanced insect resistance compared to a control plant or plant part, comprising: (a) introducing into a plant or plant part a chimeric gene or expression cassette or recombinant vector comprising a nucleic acid molecule encoding a chimeric insecticidal protein of the invention, wherein the chimeric insecticidal protein is expressed in the plant or plant part, thereby producing a plant or plant part with enhanced insect-resistance. In some aspects, the chimeric gene may encode a chimeric insecticidal protein comprising, consisting essentially of or consisting of an amino acid sequence that is at least 80%, at least 85%, at least 90%, at least 91%, at least 92%, at least 93%, at least 94%, at least 95%, at least 96%, at least 97%, at least 98%, at least 99%, or is 100% identical or similar to any of SEQ ID NOs:5-10, 18, 19 and/or 22. “Enhanced” insect resistance may be measured as any toxic effect the transgenic plant has on the insect pest that feeds on the transgenic plant. Enhanced insect resistance may be greater than 0%, at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 100%, at least 125%, at least 150%, at least 200%, at least 300%, at least 400%, at least 500%, at least 600%, at least 700%, at least 800%, at least 900%, or at least 1000% greater insecticidal activity compared to a control plant. A plant or plant part having enhance insect resistance as compared to a control plant or plant part may be produced by methods of plant transformation, plant tissue culture, or breeding. The plant or plant part may be produced by methods of sexual or asexual propagation. Any suitable control plant or plant part can be used, for example a plant of the same or similar genetic background grown in the same environment. In embodiments, the control plant or plant part is of the same genetic background and is growing in the same environment as the described plant, but it does not comprise a molecule of the invention, while the described plant does comprise a nucleic acid molecule of the invention.

In some embodiments, the invention encompasses a method of controlling an insect pest comprising, delivering to the insect pest or an environment thereof an effective amount of a chimeric insecticidal protein of the invention. In other embodiments, the chimeric protein is delivered through a transgenic plant or by topical application of an insecticidal composition comprising the chimeric insecticidal protein. In other embodiments, the chimeric protein comprises an amino acid sequence of any of SEQ ID NOs:5-10, 18, 19, or 22.

In other aspects of a method of controlling an insect pest, the transgenic plant or the insecticidal composition comprises a second insecticidal agent different from the chimeric insecticidal protein. In still other aspects, the second insecticidal agent is a protein, a dsRNA or a chemical. In further aspects, the protein is selected from the group consisting of a Cry protein, a Vip protein, a patatin, a protease, a protease inhibitor, a urease, an alpha-amylase inhibitor, a pore-forming protein, a lectin, an engineered antibody or antibody fragment, or a chitinase; or the chemical is a carbamate, a pyrethroid, an organophosphate, a friprole, a neonicotinoid, an organochloride, a nereistoxin, or a combination thereof; or the chemical comprises an active ingredient selected from the group consisting of carbofuran, carbaryl, methomyl, bifenthrin, tefluthrin, permethrin, cyfluthrin, lambda-cyhalothrin, cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, and a combination thereof.

In other aspects of the method of controlling an insect pest, the insect pest is a coleopteran insect pest. In other aspects, the coleopteran insect pest is a Diabrotica species. In still other aspects, the Diabrotica species is selected from the group consisting of Diabrotica virgifera virgifera (western corn rootworm), Diabrotica barberi (northern corn rootworm), Diabrotica undecimpunctata howardi (southern corn rootworm) and Diabrotica zeae (Mexican corn rootworm).

In some embodiments, the invention encompasses a method of reducing resistance development in a Diabrotica insect population to a chimeric insecticidal protein of the invention, the method comprising expressing in a transgenic plant fed upon by the Diabrotica insect population the chimeric insecticidal protein and an interfering RNA molecule which inhibits expression of a target gene in a larval and/or adult Diabrotica insect, and/or a Cry protein that is toxic to a Diabrotica insect pest, thereby reducing resistance development in the Diabrotica insect population compared to a Diabrotica insect population exposed only to the chimeric insecticidal protein.

In some embodiments, the invention encompasses a method of providing a corn grower with a means of controlling a Diabrotica insect pest population in a corn crop comprising (a) selling or providing to the grower transgenic corn seed that comprises a nucleic acid molecule of the invention; and (b) advertising to the grower that the transgenic corn seed produce transgenic corn plants that control a Diabrotica pest population.

In some embodiments, the invention encompasses a method of making a chimeric insecticidal protein comprising fusing in an N-terminal to C-terminal direction an N-terminal region comprising, consisting essentially of or consisting of an amino acid sequence that corresponds to amino acid 1 to amino acid 338, 339, 340, 341, 342, 343, 344, 345, 346, 347, 348, 349, 350, 351 or 352 of (i) SEQ ID NO:1 or an amino acid sequence that has at least 80% identity to SEQ ID NO:1; or (ii) SEQ ID NO:2 or an amino acid sequence that has at least 80% identity to SEQ ID NO:2; or (iii) SEQ ID NO:3 or an amino acid sequence that has at least 80% identity to SEQ ID NO:3; or (iv) SEQ ID NO:4 or an amino acid sequence that has at least 80% identity to SEQ ID NO:4, fused to (b) a C-terminal region comprising, consisting essentially of or consisting of an amino acid sequence that corresponds to amino acid 339, 340, 341, 342, 343, 344, 345, 346, 347, 348, 349, 350, 351, 352 or 353 to amino acid 488, 489 or 490 of (i) SEQ ID NO:1, or an amino acid sequence that has at least 80% identity to SEQ ID NO:1; or (ii) SEQ ID NO:2 or an amino acid sequence that has at least 80% identity to SEQ ID NO:2; or (iii) SEQ ID NO:3 or an amino acid sequence that has at least 80% identity to SEQ ID NO:3, or comprises SEQ ID NO:3.

In other embodiments, the invention encompasses a method of making a chimeric insecticidal protein comprising fusing in an N-terminal to C-terminal direction an N-terminal region comprising, consisting essentially of or consisting of an amino acid sequence that corresponds to amino acid 1 to about amino acid 363 of SEQ ID NO:17, or an amino acid sequence that has at least 80% identity to at least 99% identity to SEQ ID NO:17, fused to (a) a C-terminal region comprising, consisting essentially of or consisting of an amino acid sequence that corresponds to about amino acid 347 to about amino acid 489 of SEQ ID NO:1, or an amino acid sequence that has at least 80% identity to least 99% identity to SEQ ID NO:1; or (b) a C-terminal region comprising, consisting essentially of or consisting of an amino acid sequence that corresponds to about amino acid 346 to about amino acid 488 of SEQ ID NO:2 or an amino acid sequence that has at least 80% identity to at least 99% identity to SEQ ID NO:2.

In other embodiments, the invention provides a method of making a chimeric protein comprising an amino acid sequence of any of SEQ ID NOs:5-10, 18, 19 or 22.

### EXAMPLES

Embodiments of this invention can be better understood by reference to the following examples. The foregoing and following description of embodiments of the invention and the various embodiments are not intended to limit the claims, but are rather illustrative thereof. Therefore, it will be understood that the claims are not limited to the specific details of these examples. It will be appreciated by those skilled in the art that other embodiments of the invention may be practiced without departing from the spirit and the scope of the disclosure, the scope of which is defined by the appended claims. Art recognized recombinant DNA and molecular cloning techniques may be found in, for example, J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1. Insecticidal Activity of SproCRW and SplyCRW Chimeras

To determine if SproCRW and SplyCRW have a domain critical for insecticidal activity against corn rootworm, reciprocal chimeras were produced. The first chimeric protein, designated SproCRW/SplyCRW, comprises in an amino- to carboxy-terminal direction an N-terminal region of a SproCRW protein comprising amino acids 1-346 of SEQ ID NO: 1 joined to a C-terminal region of a SplyCRW protein comprising amino acids 346-488 of SEQ ID NO:2. The amino acid sequence of the SproCRW/SplyCRW hybrid toxin is represented by SEQ ID NO:5 (SproCRW domain is amino acids 1-346 and SplyCRW domain is amino acids 347-489). The second chimeric protein, designated SplyCRW/SproCRW, comprises in an amino- to carboxy-terminal direction an N-terminal region of an SplyCRW protein comprising amino acids 1-345 of SEQ ID NO:2 joined to a C-terminal region of an SproCRW protein comprising amino acids 346-489 of SEQ ID NO:1. The amino acid sequence of the SproCRW/SplyCRW hybrid toxin is represented by SEQ ID NO:5 (SplyCRW domain is amino acids 1-345 and SproCRW domain is amino acids 346-488). An alignment of the SproCRW/SplyCRW hybrid toxin is shown in Table 1.

TABLE 1
Alignment of SproCRW/SplyCRW hybrid toxin with parent proteins.

| | Pos | Sequence | Start | End | % Identity |
|---|---|---|---|---|---|
| Ref | 1 | SproCRW/SplyCRW (SEQ ID NO: 5) | 1 | 489 | |
| | 2 | SproCRW (SEQ ID NO: 1) | 1 | 489 | 94 |
| | 3 | SplyCRW (SEQ ID NO: 2) | 1 | 488 | 88 |

| | | |
|---|---|---|
| (SEQ ID NO: 5) | 1 | MKIESSKVEGLFSSSFTRVNAVPLPSDTLPGVGIIGCGYNPFLAYADASA |
| (SEQ ID NO: 1) | 1 | .................................................. |
| (SEQ ID NO: 2) | 1 | ..F..L...N.....L..I..I...N.A...................S.. |
| (SEQ ID NO: 5) | 51 | VLHPILDWSKSQFNEITMNGQQYQLPDVLQAVWLSNQSYASVTGKSLQSY |
| (SEQ ID NO: 1) | 51 | .................................................. |
| (SEQ ID NO: 2) | 51 | ....V........HTV.....T....EI.N.......T.S..S....... |
| (SEQ ID NO: 5) | 101 | LTELANSIKVSGNYGFFSASATNEFSDSSLRKSENEFSRCQQSFDLWSIS |
| (SEQ ID NO: 1) | 101 | .................................................. |
| (SEQ ID NO: 2) | 101 | ....S....................T........................ |
| (SEQ ID NO: 5) | 151 | IPADIARLQNYVSDDFIKLINAINPESKDSIATVFNVYGSHVLMSGVMGG |
| (SEQ ID NO: 1) | 151 | .................................................. |
| (SEQ ID NO: 2) | 151 | A..........I....K...SS.D.NNQQTL..I.......I........ |
| (SEQ ID NO: 5) | 201 | KAHVSASANKLTLTQKFEMSTIVQAKYEQLTSQLSVEDKLKYSEAFDSFS |
| (SEQ ID NO: 1) | 201 | .................................................. |
| (SEQ ID NO: 2) | 201 | ...................................A..........E... |
| (SEQ ID NO: 5) | 251 | ESGSYTYDILGGSPSLGALVFKNNSQGSSDDNLKNWIQSISSMPVLTKFI |
| (SEQ ID NO: 1) | 251 | .................................................. |
| (SEQ ID NO: 2) | 251 | ......................V.N.-D.....RK..D...T........ |
| (SEQ ID NO: 5) | 301 | DQTSLMPVWLLCEDKTKADALKKYYDNTWSKSQMAVASLRANYIDEVTEV |
| (SEQ ID NO: 1) | 301 | ..............................................L... |
| (SEQ ID NO: 2) | 300 | .....L...T....QV......N..N....QL.....R............ |
| (SEQ ID NO: 5) | 351 | LGDNSDIPAPAGYTKVPVDLNSGAGGKFIYLCYHEAQFTPVNSKQAIVGL |
| (SEQ ID NO: 1) | 351 | ..........V......I....D....YV.............G..P..DI |
| (SEQ ID NO: 2) | 350 | .................................................. |
| (SEQ ID NO: 5) | 401 | QVLYGKQEPAPDYSRINIDLNSGANGDDVYLSYKKGDATSKEVINKITAV |
| (SEQ ID NO: 1) | 401 | .....S.M...G.IK.DV......G.EF........EP..SD........ |
| (SEQ ID NO: 2) | 400 | .................................................. |
| (SEQ ID NO: 5) | 451 | YGKDQYVPTPYGYKQIPGDLNSGAGGDFVYFCTYQGGTE |
| (SEQ ID NO: 1) | 451 | ...NE...........S....A........L........ |
| (SEQ ID NO: 2) | 450 | ....................................... |

An alignment of the SplyCRW/SproCRW hybrid toxin with the parent proteins is shown in Table 2.

TABLE 2
Alignment of SplyCRW/SproCRW hybrid toxin with parent proteins.

| | Pos | Sequence | Start | End | % Identity |
|---|---|---|---|---|---|
| Ref | 1 | SplyCRW/SproCRW (SEQ ID NO: 6) | 1 | 489 | |
| | 2 | SplyCRW (SEQ ID NO: 2) | 1 | 488 | 94 |
| | 3 | SproCRW (SEQ ID NO: 1) | 1 | 489 | 88 |

| | | |
|---|---|---|
| (SEQ ID NO: 6) | 1 | MKFESLKVENLFSSSLTRINAIPLPNDALPGVGIIGCGYNPFLAYADSSA |
| (SEQ ID NO: 2) | 1 | .................................................. |
| (SEQ ID NO: 1) | 1 | ..I..S...G.....F..V..V...S.T...................A.. |
| (SEQ ID NO: 6) | 51 | VLHPVLDWSKSQFHTVTMNGQTYQLPEILNAVWLSNQTYSSVSGKSLQSY |
| (SEQ ID NO: 2) | 51 | .................................................. |
| (SEQ ID NO: 1) | 51 | ....I........NEI.....Q....DV.Q.......S.A..T....... |
| (SEQ ID NO: 6) | 101 | LTELSNSIKVSGNYGFFSASATNEFTDSSLRKSENEFSRCQQSFDLWSIS |
| (SEQ ID NO: 2) | 101 | .................................................. |
| (SEQ ID NO: 1) | 101 | ....A....................S........................ |
| (SEQ ID NO: 6) | 151 | APADIARLQNYISDDFKKLISSIDPNNQQTLATIFNVYGSHILMSGVMGG |
| (SEQ ID NO: 2) | 151 | .................................................. |
| (SEQ ID NO: 1) | 151 | I..........V....I...NA.N.ESKDSI..V.......V........ |
| (SEQ ID NO: 6) | 201 | KAHVSASANKLTLTQKFEMSTIVQAKYEQLTSQLSAEDKLKYSEAFESFS |
| (SEQ ID NO: 2) | 201 | .................................................. |
| (SEQ ID NO: 1) | 201 | ...................................V..........D... |
| (SEQ ID NO: 6) | 251 | ESGSYTYDILGGSPSLGALVFKVNNQ-DSDDNLRKWIDSISTMPVLTKFI |
| (SEQ ID NO: 2) | 251 | ..........................-....................... |
| (SEQ ID NO: 1) | 251 | ......................N.S.GS.....KN..Q...S........ |
| (SEQ ID NO: 6) | 300 | DQTSLLPVWTLCEDQVKADALKNYYNNTWSQLQMAVARLRANYIDELTFV |
| (SEQ ID NO: 2) | 300 | ..............................................V... |
| (SEQ ID NO: 1) | 301 | .....M...L....KT......K..D....KS.....S............ |
| (SEQ ID NO: 6) | 350 | LGDNSDIPAPVGYTKVPIDLNSDAGGKYVYLCYHEAQFTPVNGKQPIVDI |
| (SEQ ID NO: 2) | 350 | ..........A......V....G....FI.............S..A..GL |
| (SEQ ID NO: 1) | 351 | .................................................. |
| (SEQ ID NO: 6) | 400 | QVLYGSQMPAPGYIKIDVDLNSGAGGEFVYLSYKKGEPTSSDVINKITAV |
| (SEQ ID NO: 2) | 400 | .....K.E...D.SR.NI......N.DD........DA..KE........ |
| (SEQ ID NO: 1) | 401 | .................................................. |
| (SEQ ID NO: 6) | 450 | YGKNEYVPTPYGYKQISGDLNAGAGGDFVYLCTYQGGTE |
| (SEQ ID NO: 2) | 450 | ...DQ...........P....S........F........ |
| (SEQ ID NO: 1) | 451 | ....................................... |

The N-terminal regions (amino acids 1-346) of the SproCRW and SplyCRW proteins have 83% identity. The C-terminal regions (amino acids 347-489 (SproCRW) and amino acids 347-488 (SplyCRW) have 79% identity. The SproCRW/SplyCRW hybrid protein (SEQ ID NO:5) has 94% sequence identity across its full-length to the SproCRW sequence (SEQ ID NO:1) and 88% identity to the SplyCRW protein (SEQ ID NO:2). The SplyCRW/SproCRW hybrid protein (SEQ ID NO:6) has 94% sequence identity across its full length to the SplyCRW protein (SEQ ID NO:2) and 88% identity to the SproCRW protein (SEQ ID NO:1). The SproCRW/SplyCRW hybrid toxin has 82% identity to the SplyCRW/SproCRW hybrid toxin.

Both chimeric proteins were tested against western corn rootworm in a diet incorporation assay as described above. SproCRW and SplyCRW were used as positive controls. The results demonstrated that both chimeric proteins were equally active against WCR compared to each other and compared to the wild-type SproCRW and SplyCRW proteins.

### Example 2. Insecticidal Activity of SproCRW, SplyCRW and WoodsCRW Chimeras

This example describes new chimeric proteins made by combining portions of SproCRW and SplyCRW with portions from another novel insecticidal protein called WoodsCRW, which is described in International application publication No. WO2018132325, published on Jul. 19, 2018, and herein incorporated by reference in its entirety.

To determine if SproCRW and SplyCRW have domains that can be combined with a domain from a WoodsCRW protein to create a chimeric insecticidal protein with activity against WCR, reciprocal chimeras were produced between SproCRW and WoodsCRW and SplyCRW and WoodsCRW. WoodsCRW has 34% and 35% sequence identity with SproCRW and SplyCRW, respectively, across the full length of their sequences. A first chimeric protein was constructed, designated SproCRW/WoodsCRW, that comprises in an amino- to carboxy-terminal direction an N-terminal region of a SproCRW protein comprising amino acids 1-345 of SEQ ID NO: 1 joined to a C-terminal region of a WoodsCRW protein comprising amino acids 346-489 of SEQ ID NO:4. The amino acid sequence of the SproCRW/WoodsCRW hybrid toxin is represented by SEQ ID NO:7 (SproCRW domain is amino acids 1-345 and WoodsCRW domain is amino acids 346-489). A second chimeric protein, designated WoodsCRW/SproCRW, comprises in an amino- to carboxy-terminal direction an N-terminal region of an WoodsCRW protein comprising amino acids 1-346 of SEQ ID NO:4 joined to a C-terminal region of an SproCRW protein comprising amino acids 346-489 of SEQ ID NO:1. The amino acid sequence of the WoodsCRW/SproCRW hybrid toxin is represented by SEQ ID NO:8 (WoodsCRW domain is amino acids 1-346 and SproCRW domain is amino acids 347-490). A third chimeric protein, designated SplyCRW/WoodsCRW, comprises in an amino- to carboxy-terminal direction an N-terminal region of a SplyCRW protein comprising amino acids 1-344 of SEQ ID NO:2 joined to a C-terminal region of a WoodsCRW protein comprising amino acids 345-489 of SEQ ID NO:4. The amino acid sequence of the SproCRW/WoodsCRW hybrid toxin is represented by SEQ ID NO:9 (SplyCRW domain is amino acids 1-344 and WoodsCRW domain is amino acids 345-489). A fourth chimeric protein, designated WoodsCRW/SplyCRW, comprises in an amino- to carboxy-terminal direction an N-terminal region of an WoodsCRW protein comprising amino acids 1-346 of SEQ ID NO:4 joined to a C-terminal region of an SplyCRW protein comprising amino acids 347-490 of SEQ ID NO:2. The amino acid sequence of the WoodsCRW/SproCRW hybrid toxin is represented by SEQ ID NO:10 (WoodsCRW domain is amino acids 1-346 and SplyCRW domain is amino acids 347-490). An alignment of a WoodsCRW/SproCRW hybrid toxin with the parent proteins is shown in Table 3.

TABLE 3
Alignment of WoodsCRW/SproCRW hybrid toxin with parent proteins.

| | Pos | Sequence | Start | End | % Identity |
|---|---|---|---|---|---|
| Ref | 1 | WoodsCRW/SproCRW (SEQ ID NO: 8) | 1 | 489 | |
| | 2 | WoodsCRW (SEQ ID NO: 4) | 1 | 490 | 82 |
| | 3 | SproCRW (SEQ ID NO: 1) | 1 | 489 | 51 |

| | | |
|---|---|---|
| (SEQ ID NO: 8) | 1 | MKSLDHVAHQNLLNEPTHHKSNTKAALMRHQENLVERYLPGVEVIGAGYN |
| (SEQ ID NO: 4) | 1 | .................................................. |
| (SEQ ID NO: 1) | 1 | ..I------ESSKV.GLFSS.F.RV----NAVP.PSDT....GI..C... |
| (SEQ ID NO: 8) | 51 | PFGVYASTDSVTVQLFDWQSAPSEPVIFN-PDYIAPKAVSVQQNDEARYT |
| (SEQ ID NO: 4) | 51 | .............................-.................... |
| (SEQ ID NO: 1) | 41 | ..LA..DASA.LHPIL..SKSQFNEITM.GQQ.QL.DVLQAVWLSNQS.A |
| (SEQ ID NO: 8) | 100 | NVSGKTINTFQKNFSLKVTVAGSYNLFSGSVSNEFSSSETRNAENEFSRI |
| (SEQ ID NO: 4) | 100 | .................................................. |
| (SEQ ID NO: 1) | 91 | S.T..SLQSYLTELANSIK.S.N.GF..A.AT....D.SL.KS......C |
| (SEQ ID NO: 8) | 150 | QQSIRVWSLRL-AYTDSLREYLKADVRDYIDSIQSDAQ--IEILFDRYGS |
| (SEQ ID NO: 4) | 150 | ...........-..........................--.......... |
| (SEQ ID NO: 1) | 141 | ...FDL..ISIP.DIAR.QN.VSD.FIKL.NA.NPESKDS.ATV.NV... |
| (SEQ ID NO: 8) | 197 | HFLTGVVMGGAAIMASSTNKVQVDHTYENETIAKASYEALTGQISAETAA |
| (SEQ ID NO: 4) | 197 | .................................................. |
| (SEQ ID NO: 1) | 191 | .V.MSG....K.HVSA.A..LTLTQKF.MS..VQ.K..Q..S.L.V.DKL |
| (SEQ ID NO: 8) | 247 | KYRQSMSSFSQNSDIHKIVVGGDGVAGAKVYSGDKA-----DFDANADTV |
| (SEQ ID NO: 4) | 247 | ....................................-----......... |
| (SEQ ID NO: 1) | 241 | ..SEAFD...ESGSYTYDIL..SPSL..L.FKNNSQGSSDDNLKN.IQSI |
| (SEQ ID NO: 8) | 292 | GTSPDFVDFVSSVPMLGIWELCKDDAQAKKMEDYYNNTWAPRKSKEAQIY |
| (SEQ ID NO: 4) | 292 | .................................................. |
| (SEQ ID NO: 1) | 291 | SSM.VLTK.IDQTSLMPV.L..E.KTK.DALKK..D...SKSQMAV.SLR |
| (SEQ ID NO: 8) | 342 | ADYIDELTFVLGDNSDIPAPVGYTKVPIDLNSDAGGKYVYLCYHEAQFTP |
| (SEQ ID NO: 4) | 342 | .....AVEVIQSNS.GVRP.S....IDY...KG...D.I.....K.RYSA |
| (SEQ ID NO: 1) | 341 | .N................................................ |
| (SEQ ID NO: 8) | 392 | VN-GKQPIVDIQVLYGSQMPAP-GYIKIDVDLNSGAGGEFVYLSYKKGEP |
| (SEQ ID NO: 4) | 392 | YSEN.DCVS.LIIIK.NGAR..S..T.......ED...KYL..C...QSY |
| (SEQ ID NO: 1) | 391 | ..-...................-........................... |
| (SEQ ID NO: 8) | 440 | TSSDVINKITAVYGKNEYVPTPYGYKQISGDLNAGAGGDFVYLCTYQGGT |
| (SEQ ID NO: 4) | 442 | DNVEA.KGLAV.G.D.SHT.A....RR.DT.V.E....EYI.I.YSK.A- |
| (SEQ ID NO: 1) | 439 | .................................................. |
| (SEQ ID NO: 8) | 490 | E |
| (SEQ ID NO: 4) | 491 | - |
| (SEQ ID NO: 1) | 489 | . |

An alignment of a WoodsCRW/SplyCRW hybrid toxin with the parent proteins is shown in Table 4.

TABLE 4
Alignment of WoodsCRW/SplyCRW hybrid toxin with parent proteins.

| | Pos | Sequence | Start | End | % Identity |
|---|---|---|---|---|---|
| Ref | 1 | WoodsCRW/SplyCRW (SEQ ID NO: 10) | 1 | 489 | |
| | 2 | WoodsCRW (SEQ ID NO: 4) | 1 | 490 | 81 |
| | 3 | SplyCRW (SEQ ID NO: 2) | 1 | 489 | 53 |

| | | |
|---|---|---|
| (SEQ ID NO: 10) | 1 | MKSLDHVAHQNLLNEPTHHKSNTKAALMR-HQENLVERYLPGVEVIGAGY |
| (SEQ ID NO: 4) | 1 | .................................................. |
| (SEQ ID NO: 2) | 1 | ..-FESLKVE..FS----------SS.T.INAIP.PNDA....GI..C.. |
| (SEQ ID NO: 10) | 50 | NPFGVYASTDSVIVQLFDWQSAPSEPVIFNPD-YIAPKAVSVQQNDEARY |
| (SEQ ID NO: 4) | 50 | ................................-................. |
| (SEQ ID NO: 2) | 40 | ...LA..DSSA.LHPVL..SKSQFHT.TM.GQT.QL.EILNAVWLSNQT. |
| (SEQ ID NO: 10) | 99 | TNVSGKTINTFQKNFSLKVTVAGSYNLFSGSVSNEFSSSETRNAENEFSR |
| (SEQ ID NO: 4) | 99 | .................................................. |
| (SEQ ID NO: 2) | 90 | SS....SLQSYLTEL.NSIK.S.N.GF..A.AT...TD.SL.KS...... |
| (SEQ ID NO: 10) | 149 | IQQSIRVWSLRL-AYTDSLREYLKADVRDYIDSIQSDAQ--IEILFDRYG |
| (SEQ ID NO: 4) | 149 | ............-..........................--......... |
| (SEQ ID NO: 2) | 140 | C...FDL..ISAP.DIAR.QN.ISD.FKKL.S..DPNN.QTLATI.NV.. |
| (SEQ ID NO: 10) | 196 | SHFLIGVVMGGAAIMASSINKVQVDHIYENETIAKASYEALTGQISAETA |
| (SEQ ID NO: 4) | 196 | .................................................. |
| (SEQ ID NO: 2) | 190 | ..I.MSG....K.HVSA.A..LTLIQKF.MS..VQ.K..Q..S.L...DK |
| (SEQ ID NO: 10) | 246 | AKYRQSMSSFSQNSDIHKIVVGGDGVAGAKVYSGDKADFD----AWADTV |
| (SEQ ID NO: 4) | 246 | ........................................----...... |
| (SEQ ID NO: 2) | 240 | L..SEAFE...ESGSYTYDIL..SPSL..L.FKVNNQ.S.DNLRK.I.SI |
| (SEQ ID NO: 10) | 292 | GTSPDFVDFVSSVPMLGIWELCKDDAQAKKMEDYYNNTWAPRKSKEAQIY |
| (SEQ ID NO: 4) | 292 | .................................................. |
| (SEQ ID NO: 2) | 290 | S.M.VLTK.IDQTSL.PV.T..E.QVK.DALKN......SQLQMAV.RLR |
| (SEQ ID NO: 10) | 342 | ADYIDEVIFVLGDNSDIPAPAGYTKVPVDLNSGAGGKFIYLCYHEAQFTP |
| (SEQ ID NO: 4) | 342 | .....A.EVIQSNS.GVRP.S....IDY...K....DY......K.RYSA |
| (SEQ ID NO: 2) | 340 | .N................................................ |
| (SEQ ID NO: 10) | 392 | VN-SKQAIVGLQVLYGKQEPAPD-YSRINIDLNSGANGDDVYLSYKKGDA |
| (SEQ ID NO: 4) | 392 | YSEN.DCVSD.IIIK.NGAR..SG.TK.DV...ED.G.KYL..C...QSY |
| (SEQ ID NO: 2) | 390 | ..-....................-.......................... |
| (SEQ ID NO: 10) | 440 | TSKEVINKITAVYGKDQYVPTPYGYKQIPGDLNSGAGGDFVYFCTYQGGT |
| (SEQ ID NO: 4) | 442 | DNV.A.KGLAV.G.DNSHT.A....RR.DT.V.E....EYI.I.YSK.A- |
| (SEQ ID NO: 2) | 438 | .................................................. |
| (SEQ ID NO: 10) | 490 | E |
| (SEQ ID NO: 4) | 491 | - |
| (SEQ ID NO: 2) | 488 | . |

The SproCRW/WoodsCRW chimeric protein (SEQ ID NO:7) has 94% sequence identity across its full-length to the SproCRW sequence (SEQ ID NO:1) and 88% identity to the SplyCRW protein (SEQ ID NO:2). The SplyCRW/SproCRW hybrid protein (SEQ ID NO:23) has 94% sequence identity across its full length to the SplyCRW protein (SEQ ID NO:2) and 88% identity to the SproCRW protein (SEQ ID NO:1).

All four chimeric proteins were prepared for testing against WCR as described above. The SproCRW/WoodsCRW chimeric protein and the SplyCRW/WoodsCRW protein were completely insoluble. The WoodsCRW/SproCRW (SEQ ID NO:8) and WoodsCRW/SplyCRW (SEQ ID NO:10) chimeric proteins were tested against western corn rootworm in a diet incorporation assay as described above. SproCRW and SplyCRW were used as positive controls. Results are shown in Table 5. Percent mortality and growth inhibition, where s=small larvae, m=medium larvae and 1=large larvae, were taken 3 and 6 days post-infestation. The results demonstrated that both chimeric proteins were equally active against WCR compared to each other and compared to the wild-type SproCRW and SplyCRW proteins.

TABLE 5
Insecticidal activity of chimeric proteins against WCR.

| | Day 3 | | Day 6 | |
|---|---|---|---|---|
| Treatment | % Mort | Growth | % Mort | Growth |
| pET29a-empty | 0 | l | 8 | l |
| WoodsCRW-SproCRW | 33 | m | 92 | m/l |
| WoodsCRW-SplyCRW | 33 | m | 92 | m |

### Example 3. Insecticidal Activity of Plu1415CRW, SproCRW and SplyCRW Chimeras

This example describes chimeric proteins made by combining portions of SproCRW and SplyCRW with portions from a non-insecticidal protein called Plu1415 (SEQ ID NO:17), which is described in International Application Publication No. WO2018132325, published on Jul. 19, 2018, herein incorporated by reference in its entirety. Plu1415, a protein from the bacteria Photorhabdus luminescens does not have any activity against Diabrotica insect pests. The crystal structure of Plu1415 is described by Rosado et al., 2007 (Science, 317:1548-1551). Like Plu1415, SporCRW and SplyCRW are comprised of an N-terminal MACPF domain and a C-terminal β-prism domain. The β-prism domain of Plu1415 is from about amino acid position 364 to about amino acid position 510 of SEQ ID NO:17. The β-prism domain of SproCRW is from about amino acid position 347 to about amino acid position 489 of SEQ ID NO:1, and for SplyCRW from about amino acid position 346 to about amino acid position 488 of SEQ ID NO:2. The overall percent identity between Plu1415 and SproCRW and Plu1415 and SplyCRW is 25% and 26%, respectively. There is even lower identity in the β-prism domains of Plu1415 and SproCRW and SplyCRW with 21% and 23%, respectively.

Two chimeric proteins were made to determine whether the β-prism domains of SproCRW or SplyCRW could confer insecticidal activity to a non-insecticidal protein, i.e. Plu1415. A Plu1415-SproCRW chimera (SEQ ID NO:18) comprises the N-terminal MACPF domain of Plu1415 (amino acids 1-363 of SEQ ID NO:17) and the β-prism domain of SproCRW (amino acids 347-489 of SEQ ID NO:1). Similarly, the Plu1415-SplyCRW chimera (SEQ ID NO:19) comprises the N-terminal MACPF domain of Plu1415 (amino acids 1-363 of SEQ ID NO:17) and the β-prism domain of SplyCRW (amino acids 346-488 of SEQ ID NO:2). Alignments of a Plu1415/SproCRW chimeric protein and a Plu1415/SplyCRW chimeric protein with their respective parent proteins are shown in Tables 6 and 7, where a “.” below an amino acid position indicates the same amino acid.

TABLE 6
Alignment of Plu1415/SproCRW hybrid protein with parent proteins.

| | Pos | Sequence | Start | End | % Identity |
|---|---|---|---|---|---|
| Ref | 1 | P1u1415/SproCRW (SEQ ID NO: 18) | 1 | 506 | |
| | 2 | Plu1415 (SEQ ID NO: 17) | 1 | 510 | 77 |
| | 3 | SproCRW (SEQ ID NO: 1) | 1 | 489 | 46 |

| | | |
|---|---|---|
| SEQ ID NO: 18 | 1 | msndktgksleqenserdveirdrnyfrkls---lfddtviagaemigts |
| SEQ ID NO: 17 | 1 | ...............................---................ |
| SEQ ID NO: 1 | 1 | ---------mki.s.k--..glfsss.trvnavp.ps..-lp.vgi..cg |
| SEQ ID NO: 18 | 48 | ydvfgkycnvgscmnslfderkinasednfkkvtilgktlkvpyyidcys |
| SEQ ID NO: 17 | 48 | .................................................. |
| SEQ ID NO: 1 | 39 | .np.la.adasavlhpil.-----w.ksq.nei.mn.qqyql.dvlqavw |
| SEQ ID NO: 18 | 98 | vgdlkytnasgesiesyqsnissksrikgnylffsaslkvdfdtdsltdf |
| SEQ ID NO: 17 | 98 | .................................................. |
| SEQ ID NO: 1 | 84 | lsnqs.asvt.k.lq..ltelansikvs...g.....atne.sds..rks |
| SEQ ID NO: 18 | 148 | enafsriqytydlyilkssaea--lkeflkesvktaldkadte--edmnd |
| SEQ ID NO: 17 | 148 | ......................--...................--..... |
| SEQ ID NO: 1 | 134 | ..e...c.qsf..wsisip.diar.qnyvsddfiklinainp.skdsiat |
| SEQ ID NO: 18 | 194 | lfntwgshflsgvvmggcaqyssstnkytsnitnsfdv--vaaasfagfi |
| SEQ ID NO: 17 | 194 | ......................................--.......... |
| SEQ ID NO: 1 | 184 | v..vy...v.msg....k.hv.a.a..l.--..qk.emstivq.kyeqlt |
| SEQ ID NO: 18 | 242 | g-lsartgnsfmedikkfrsasnikthaiggdlsrfdpfggatsadqpsa |
| SEQ ID NO: 17 | 242 | .-................................................ |
| SEQ ID NO: 1 | 232 | sq..vedklkys.afds.sesgsytydil..-----s.sl..lvfknn.q |
| SEQ ID NO: 18 | 291 | eeiaaakkafedwkasvpnapelvnfadsnpltgiwelcsdrtqkaklkk |
| SEQ ID NO: 17 | 291 | .................................................. |
| SEQ ID NO: 1 | 277 | ---gssddnlkn.iq.issm.v.tk.i.qts.mpv.l..e.k.kada... |
| SEQ ID NO: 18 | 341 | hfetvwapaesakrrvhadyideltfvlgdnsdipapvgyt--kvpidln |
| SEQ ID NO: 17 | 341 | .......................i--ii.i.ntntp.e..igl.stk.e. |
| SEQ ID NO: 1 | 324 | yydnt.sksqm.vaslr.n......................--....... |
| SEQ ID NO: 18 | 389 | sdaggkyvylcyheaqftp-vngkqpivdiq-vlygsqmpapgyikidvd |
| SEQ ID NO: 17 | 389 | lnsk.n-ic.fm.k.kyd.nidn.dc.telkfitvrdks.egdwv..pq. |
| SEQ ID NO: 1 | 372 | ...................-...........-.................. |
| SEQ ID NO: 18 | 437 | lnsgaggefvylsykkgeptssdvinkitavygk-neyvptpygykqisg |
| SEQ ID NO: 17 | 438 | iyi-spnqyl..c.lpakysaeka.kd.qllcsscgssmil....ndvld |
| SEQ ID NO: 1 | 420 | ..................................-............... |
| SEQ ID NO: 18 | 486 | dlnagagg---dfvylctyqggte |
| SEQ ID NO: 17 | 487 | erger.nated.n.hyli.sa.wk |
| SEQ ID NO: 1 | 469 | ........---............. |

TABLE 7
Alignment of Plu1415/SplyCRW hybrid protein with parent proteins.

| | Pos | Sequence | Start | End | % Identity |
|---|---|---|---|---|---|
| Ref | 1 | P1u1415/SplyCRW (SEQ ID NO: 19) | 1 | 506 | |
| | 2 | Plu1415 (SEQ ID NO: 17) | 1 | 510 | 77 |
| | 3 | SplyCRW (SEQ ID NO: 2) | 1 | 488 | 47 |

| | | |
|---|---|---|
| SEQ ID NO: 19 | 1 | msndktgksleqenserdveirdrnyfrklslfddtviagaemigtsydv |
| SEQ ID NO: 17 | 1 | .................................................. |
| SEQ ID NO: 2 | 1 | ----mkfe..kv..lfssslt.----inaip.pn.a-lp.vgi..cg.np |
| SEQ ID NO: 19 | 51 | fgkycnvgscmnslfderkinasednfkkvtilgktlkvpyyidcysvgd |
| SEQ ID NO: 17 | 51 | .................................................. |
| SEQ ID NO: 2 | 42 | .la.adssavlhpvl.-----w.ksq.ht..mn.q.yql.eilnavwlsn |
| SEQ ID NO: 19 | 101 | lkytnasgesiesyqsnissksrikgnylffsaslkvdfdtdsltdfena |
| SEQ ID NO: 17 | 101 | .................................................. |
| SEQ ID NO: 2 | 87 | qt.ssv..k.lq..ltel.nsikvs...g.....atne.tds..rks..e |
| SEQ ID NO: 19 | 151 | fsriqytydlyilkssaea--lkeflkesvktaldkad--teedmnd1fn |
| SEQ ID NO: 17 | 151 | ...................--............................. |
| SEQ ID NO: 2 | 137 | ...c.qsf..wsisap.diar.qnyisddf.klissi.pnnqqtlati.. |
| SEQ ID NO: 19 | 197 | twgshflsgvvmggcaqyssstnkytsnitnsfdv--vaaasfagfig-l |
| SEQ ID NO: 17 | 197 | ...................................--...........-. |
| SEQ ID NO: 2 | 187 | vy...i.msg....k.hv.a.a..l.--..qk.emstivq.kyeqltsq. |
| SEQ ID NO: 19 | 244 | sartgnsfmedikkfrsasnikthaiggdlsrfdpfggatsadqpsaeei |
| SEQ ID NO: 17 | 244 | .................................................. |
| SEQ ID NO: 2 | 235 | ..edklkys.afes.sesgsytydil..-----s.sl..lvekvnnqdsd |
| SEQ ID NO: 19 | 294 | aaakkafedwkasvpnapelvnfadsnpltgiwelcsdrtqkaklkkhfe |
| SEQ ID NO: 17 | 294 | .................................................. |
| SEQ ID NO: 2 | 280 | dnlr.----.id.istm.v.tk.i.qts.lpv.t..e.qvkada..nyyn |
| SEQ ID NO: 19 | 344 | tvwapaesakrrvhadyidevtfvlgdnsdipapagyt--kvpvdlnsga |
| SEQ ID NO: 17 | 344 | ....................i--ii.i.ntntp.e..igl.stk.e.lns |
| SEQ ID NO: 2 | 326 | nt.sqlqm.va.lr.n......................--.......... |
| SEQ ID NO: 19 | 392 | ggkfiylcyheaqftp-vnskqaivglqvlygkqe-papdysrinidlns |
| SEQ ID NO: 17 | 392 | k.n-.c.fm.k.kyd.nidn.dc.te.kfitvrdks.eg.wvk.pq.iyi |
| SEQ ID NO: 2 | 374 | ................-..................-.............. |
| SEQ ID NO: 19 | 440 | gangddvylsykkgdatskevinkitavygk-dqyvptpygykqi---pg |
| SEQ ID NO: 17 | 441 | sp.-gyl..c.lpakysaeka.kd.qllcsscgssmil....ndvlder. |
| SEQ ID NO: 2 | 422 | ...............................-.............---.. |
| SEQ ID NO: 19 | 486 | dlnsgaggdfvyfctyqggte |
| SEQ ID NO: 17 | 490 | eranated.n.hyli.sa.wk |
| SEQ ID NO: 2 | 468 | ..................... |

A third chimeric protein was made as a control whereby the β-prism domain of Plu1415 was replaced with a β-prism domain of a HmassCRW protein, which is described in International Application Publication No. WO2018081194, published on May 3, 2018. The three chimeras were tested for insecticidal activity against western corn rootworm (WCR; Diabrotica virgifera) using a diet incorporation assay as described above.

Results of the WCR assay, shown in Table 8, demonstrate that the SplyCRW β-prism domain can confer insecticidal activity to a non-insecticidal protein (Plu1415) by replacing the non-insecticidal protein's β-prism domain (i.e. from about amino acid position 364-510 of SEQ ID NO:17) with the SplyCRW β-prism domain (from about amino acid position 346-488 of SEQ ID NO:2).

TABLE 8
Insecticidal activity of chimeric proteins.

| | Day 3 | | Day 6 | |
|---|---|---|---|---|
| Treatment | % Mort | Growth | % Mort | Growth |
| pET29a-empty | 0 | l | 17 | l |
| Plul415-SproCRW | 8 | l | 17 | l |
| Plu 1415-SplyCRW | 50 | m | 100 | — |
| Plul415-HmassCRW | 0 | L | 17 | l |

### Example 4. Transformation of Maize with Hybrid Protein

A maize optimized nucleotide sequence that encodes a chimeric insecticidal protein of the invention, for example SproCRW/SplyCRW (SEQ ID NO:5), is generated as described in U.S. Pat. No. 6,051,760, herein incorporated by reference.

Two plant expression cassettes are constructed to introduce the SproCRW/SplyCRW coding sequence into maize. The first cassette comprises a maize ubiquitin 1 (Ubi1) promoter operably linked to the SproCRW/SplyCRW coding sequence which is operably linked to a maize Ubi361 terminator. The second cassette comprises a maize Ubi1 promoter operably linked to a pmi coding sequence that encodes the selectable marker phosphomannose isomerase (PMI), which is operably linked to a maize Ubi1 terminator. A recombinant plant transformation binary vector comprising the two expression cassettes is generated for maize transformation experiments.

The binary vector is transformed into Agrobacterium tumefaciens using standard molecular biology techniques. To prepare the Agrobacteria for transformation, cells are cultured in liquid YPC media at 28° C. and 220 rpm overnight.

Agrobacterium transformation of immature maize embryos is performed essentially as described in Negrotto et al., 2000, Plant Cell Reports 19: 798-803. For this example, all media constituents are essentially as described in Negrotto et al., supra. However, various media constituents known in the art may be substituted.

Briefly, Agrobacterium strain LBA4404 (pSB1) containing the binary vector plant transformation vector is grown on YEP (yeast extract (5 g/L), peptone (10 g/L), NaCl (5 g/L), 15 g/l agar, pH 6.8) solid medium for 2-4 days at 28° C. Approximately 0.8×10⁹ Agrobacterium are suspended in LS-inf media supplemented with 100 μM As (Negrotto et al., supra). Bacteria are pre-induced in this medium for 30-60 minutes.

Immature embryos from a suitable genotype are excised from 8-12 day old ears into liquid LS-inf+100 μM As. Embryos are rinsed once with fresh infection medium. Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/l) and silver nitrate (1.6 mg/l) and cultured in the dark for 28° C. for 10 days.

Immature embryos, producing embryogenic callus are transferred to LSD1M0.5S medium. The cultures are selected on this medium for about 6 weeks with a subculture step at about 3 weeks. Surviving calli are transferred to Reg1 medium supplemented with mannose. Following culturing in the light (16 hour light/8 hour dark regiment), green tissues are then transferred to Reg2 medium without growth regulators and incubated for about 1-2 weeks. Plantlets are transferred to Magenta GA-7 boxes (Magenta Corp, Chicago Ill.) containing Reg3 medium and grown in the light.

Following transformation, selection, and regeneration, plants are assayed for the presence of the pmi gene and the SproCRW/SplyCRW maize codon-optimized coding sequence using TaqMan® analysis. Plants are also tested for the presence of the vector backbone. Transgenic maize plants negative for the vector backbone and comprising one copy of the transgene from the binary vector are transferred to a greenhouse and tested for insecticidal activity against WCR.

### Example 5: Chimeric Insecticidal Proteins in Combination with a Second Insecticidal Agent

SproCRW/SplyCRW protein is combined with a double stranded RNA (dsRNA) against an essential target and known to have insecticidal activity is prepared. In non-limiting examples, the dsRNA may target a gene encoding vacuolar ATP synthase, beta-tubulin, 26S proteosome subunit p28 protein, EF1α 48D, troponin I, tetraspanin, clathrin heavy chain, gamma-coatomer, beta-coatomer, and/or juvenile hormone epoxide hydrolase (PCT Patent Application Nos. PCT/US17/044825; PCT/US17/044831; PCT/US17/044832; U.S. Pat. No. 7,812,219; each herein incorporated by reference). The dsRNA and purified protein are tested for efficacy against WCR in a diet-incorporation assay, performed essentially as described in Example 1.

### Example 6. Genome Editing in Plant Cells In Situ to Generate Modified Chimeric Proteins

The following Example illustrates the use of genome editing of a plant cell genome in situ to incorporate mutations, to make the chimeric proteins described herein (including but not limited to the chimeric proteins described in Examples 1 and 2) into a coding sequence for a wild-type Serratia insecticidal protein or a Woods insecticidal protein, including SproCRW (SEQ ID NO: 1), SplyCRW (SEQ ID NO:2) and/or SquiCRW (SEQ ID NO:3) or into a coding sequence for an already modified SproCRW, SplyCRW and/or SquiCRW protein.

Targeted genome modification, also known as genome editing, is useful for introducing mutations in specific DNA sequences. These genome editing technologies, which include zinc finger nucleases (ZNFs), transcription activator-like effector nucleases (TALENS), meganucleases and clustered regularly interspaced short palindromic repeats (CRISPR) have been successfully applied to over 50 different organisms including crop plants. See, e.g., Belhaj, K., et al., Plant Methods 9, 39 (2013); Jiang, W., et al., Nucleic Acids Res, 41, e188 (2013)). The CRISPR/Cas system for genome editing is based on transient expression of Cas9 nuclease and an engineered single guide RNA (sgRNA) that specifies the targeted polynucleotide sequence.

Cas9 is a large monomeric DNA nuclease guided to a DNA target sequence with the aid of a complex of two 20-nucleotide (nt) non-coding RNAs: CRIPSR RNA (crRNA) and trans-activating crRNA (tracrRNA), which are functionally available as single synthetic RNA chimera. The Cas9 protein contains two nuclease domains homologous to RuvC and HNH nucleases. The HNH nuclease domain cleaves the complementary DNA strand, whereas the RuvC-like domain cleaves the non-complementary strand and, as a result, a blunt cut is introduced in the target DNA.

When the Cas9 and the sgRNA are transiently expressed in living maize cells, double strand breaks (DSBs) in the specific targeted DNA is created in the transgenic maize cell. Mutation at the break site is introduced through the non-homologous end joining and homology-directed DNA repair pathways.

Specific mutations are introduced into a coding sequence for the native SproCRW insecticidal protein (SEQ ID NO: 1) or a modified SproCRW protein, through the use of recombinant plasmids expressing the Cas9 nuclease and the sgRNA target that is maize codon optimized for the SproCRW or modified SproCRW sequence in the transgenic maize. Implementation of the method is by an agroinfiltration method with Agrobacterium tumefaciens carrying the binary plasmid harboring the specified target sequence of interest. After the sgRNA binds to the target SproCRW or modified SproCRW coding sequence, the Cas9 nuclease makes specific cuts into the coding sequence and introduces the desired mutation(s) during DNA repair, for example introducing the C-termainal portion of a SplyCRW protein. Thus, a now mutated SproCRW coding sequence will encode the SproCRW/SplyCRW chimeric protein.

Plant cells comprising the genome edited SproCRW/SplyCRW coding sequences are screened by PCR and sequencing. Calli that harbor genome edited SproCRW/SplyCRW or modified SproCRW/SplyCRW coding sequences are induced to regenerate plants for phenotype evaluation for insecticidal activity of the expressed SproCRW/SplyCRW chimeric protein against WCRW, Northern Corn Rootworm (Diabrotica barberi), Southern Corn Rootworm (Diabrotica undecimpunctata howardi) and/or Mexican Corn Rootworm (Diabrotica virgifera zeae).

It should be understood that the examples and embodiments described herein are for illustrative purposes only and that various modifications or changes in light thereof of the description will be suggested to persons skilled in the art and are to be included within the spirit and purview of this application and the scope of the appended claims.

All publications and patent applications mentioned in this specification are indicative of the level of skill of those skilled in the art that this invention pertains. All publications and patent applications are herein incorporated by reference to the same extent as if each individual publication or patent application was specifically and individually indicated to be incorporated by reference.

1. A chimeric insecticidal protein that is toxic to an insect pest, comprising in an N-terminal to C-terminal direction (a) an N-terminal region comprising an amino acid sequence that corresponds to amino acid 1 to amino acid 338, 339, 340, 341, 342, 343, 344, 345, 346, 347, 348, 349, 350, 351 or 352 of (i) SEQ ID NO:1 or an amino acid sequence that has at least 80% identity to SEQ ID NO:1; or (ii) SEQ ID NO:2 or an amino acid sequence that has at least 80% identity to SEQ ID NO:2; or (iii) SEQ ID NO:3 or an amino acid sequence that has at least 80% identity to SEQ ID NO:3; or (iv) SEQ ID NO:4 or an amino acid sequence that has at least 80% identity to SEQ ID NO:4, fused to (b) a C-terminal region comprising an amino acid sequence that corresponds to amino acid 339, 340, 341, 342, 343, 344, 345, 346, 347, 348, 349, 350, 351, 352 or 353 to amino acid 488, 489 or 490 of (i) SEQ ID NO:1, or an amino acid sequence that has at least 80% identity to at least 99% identity to SEQ ID NO:1; or (ii) SEQ ID NO:2 or an amino acid sequence that has at least 80% identity to at least 99% identity to SEQ ID NO:2; or (iii) SEQ ID NO:3 or an amino acid sequence that has at least 80% identity to at least 99% identity to SEQ ID NO:3, or comprises SEQ ID NO:3; or (c) an N-terminal region comprising an amino acid sequence that corresponds to amino acid 1 to about amino acid 363 of SEQ ID NO:17, fused to (i) a C-terminal region comprising an amino acid sequence that corresponds to about amino acid 347 to about amino acid 489 of SEQ ID NO:1 or an amino acid sequence having at least 80% identity to at least 99% identity to SEQ ID NO:1; or (ii) a C-terminal region comprising an amino acid sequence that corresponds to about amino acid 346 to about amino acid 488 of SEQ ID NO:2, or an amino acid sequence having at least 80% identity to at least 99% identity to SEQ ID NO:2.

2. The chimeric insecticidal protein of claim 1, wherein (a) the N-terminal region comprises an amino acid sequence that corresponds to amino acid 1 to amino acid 345 of SEQ ID NO:1 and the C-terminal region comprises an amino acid sequence that corresponds to amino acid 346 to amino acid 488 of SEQ ID NO:2; or (b) the N-terminal region comprises an amino acid sequence that corresponds to amino acid 1 to amino acid 345 of SEQ ID NO:2 and the C-terminal region comprises an amino acid sequence that corresponds to amino acid 346 to amino acid 489 of SEQ ID NO:1; or (c) the N-terminal region comprises an amino acid sequence that corresponds to amino acid 1 to amino acid 346 of SEQ ID NO:4 and the C-terminal region comprises an amino acid sequence that corresponds to amino acid 346 to amino acid 489 of SEQ ID NO:1; or (d) the N-terminal region comprises an amino acid sequence that corresponds to amino acid 1 to amino acid 346 of SEQ ID NO:4 and the C-terminal region comprises an amino acid sequence that corresponds to amino acid 346 to amino acid 488 of SEQ ID NO:2; or (e) the N-terminal region comprises an amino acid sequence that corresponds to amino acid 1 to amino acid 363 of SEQ ID NO:17 and the C-terminal region comprises an amino acid sequence that corresponds to amino acid 347 to amino acid 489 of SEQ ID NO:1, or (f) the N-terminal region comprises an amino acid sequence that corresponds to amino acid 1 to amino acid 363 of SEQ ID NO:17 and the C-terminal region comprises an amino acid sequence that corresponds to amino acid 346 to amino acid 488 of SEQ ID NO:2.

3. The chimeric insecticidal protein of claim 2, wherein the chimeric insecticidal protein comprises an amino acid sequence that has at least 80% identity to at least 99% identity to SEQ ID NO:5, SEQ ID NO:6, SEQ ID NO:7, SEQ ID NO:8, SEQ ID NO:9, SEQ ID NO:10, SEQ ID NO:18, SEQ ID NO:19 or SEQ ID NO:22.

4. The chimeric insecticidal protein of claim 1, wherein (a) the N-terminal region comprises amino acid 1 to amino acid 345 of SEQ ID NO:1 and the C-terminal region comprises amino acid 346 to amino acid 488 of SEQ ID NO:2; or (b) the N-terminal region comprises amino acid 1 to amino acid 345 of SEQ ID NO:2 and the C-terminal region comprises amino acid 346 to amino acid 489 of SEQ ID NO:1; or (c) the N-terminal region comprises amino acid 1 to amino acid 346 of SEQ ID NO:4 and the C-terminal region comprises amino acid 346 to amino acid 489 of SEQ ID NO:1; or (d) the N-terminal region comprises amino acid 1 to amino acid 346 of SEQ ID NO:4 and the C-terminal region comprises amino acid 346 to amino acid 488 of SEQ ID NO:2; or (e) the N-terminal region comprises amino acid 1 to amino acid 363 of SEQ ID NO:17 and the C-terminal region comprises amino acid 347 to amino acid 489 of SEQ ID NO:1, or (f) the N-terminal region comprises amino acid 1 to amino acid 363 of SEQ ID NO:17 and the C-terminal region comprises amino acid 346 to amino acid 488 of SEQ ID NO:2.

5. The chimeric insecticidal protein of claim 4, wherein the chimeric insecticidal protein comprises SEQ ID NO:5, SEQ ID NO:6, SEQ ID NO:7, SEQ ID NO:8, SEQ ID NO:9, SEQ ID NO:10, SEQ ID NO:18, SEQ ID NO:19 or SEQ ID NO:22.

6.-8. (canceled)

9. A nucleic acid molecule comprising (a) a nucleic acid sequence encoding the chimeric insecticidal protein of claim 1; or (b) the nucleotide sequence of (a) that is codon optimized for expression in a transgenic organism.

10.-13. (canceled)

14. The nucleic acid molecule of claim 9, wherein the nucleotide sequence comprises any of SEQ ID NOs:11-16, 20 or 21.

15. A chimeric gene comprising a heterologous promoter operably linked to the nucleic acid molecule of claim 9.

16.-21. (canceled)

22. A recombinant vector comprising the chimeric gene of claim 15.

23. A host cell comprising the recombinant vector of claim 22, wherein the host cell is a bacterial cell or plant cell.

24. The transgenic bacterial cell of claim 23, wherein the bacterial cell is in the genus Bacillus, Clostridium, Xenorhabdus, Photorhabdus, Pasteuria, Escherichia, Pseudomonas, Erwinia, Serratia, Klebsiella, Salmonella, Pasteurella, Xanthomonas, Streptomyces, Rhizobium, Rhodopseudomonas, Methylophilius, Agrobacterium, Acetobacter, Lactobacillus, Arthrobacter, Azotobacter, Leuconostoc, or Alcaligenes.

25. (canceled)

26. The transgenic plant cell of claim 23, wherein the plant cell is a dicot plant cell or a monocot plant cell.

27. The transgenic plant cell of claim 26, wherein (a) the dicot plant cell is selected from the group consisting of a soybean cell, sunflower cell, tomato cell, cole crop cell, cotton cell, sugar beet cell and tobacco cell; or (b) the monocot plant cell is selected from the group consisting of a barley cell, maize cell, oat cell, rice cell, sorghum cell, sugar cane cell and wheat cell.

28. A transgenic plant or plant part comprising the transgenic plant cell of claim 27.

29. (canceled)

30. An insecticidal composition comprising the chimeric insecticidal protein of claim 1 and an agriculturally acceptable carrier.

31.-37. (canceled)

38. A method of producing a transgenic plant or plant part having enhanced insect resistance compared to a control plant or plant part, comprising: (a) introducing into a plant or plant part the chimeric gene of claim 15, wherein the insecticidal protein is expressed in the plant or plant part, thereby producing a plant or plant part with enhanced insect-resistance.

39. (canceled)

40. A method of controlling an insect pest comprising, delivering to the insect pest or an environment thereof an effective amount of the chimeric insecticidal protein of claim 1.

41.-44. (canceled)

45. The method of claim 40, wherein the insect pest is a coleopteran insect pest.

46. The method of 45, wherein the coleopteran insect pest is a Diabrotica species.

47.-49. (canceled)

50. A method of making a chimeric insecticidal protein comprising fusing in an N-terminal to C-terminal direction (a) an N-terminal region comprising an amino acid sequence that corresponds to amino acid 1 to amino acid 338, 339, 340, 341, 342, 343, 344, 345, 346, 347, 348, 349, 350, 351 or 352 of (i) SEQ ID NO:1 or an amino acid sequence that has at least 80% identity to at least 99% identity to SEQ ID NO:1; or (ii) SEQ ID NO:2 or an amino acid sequence that has at least 80% identity of at least 99% identity to SEQ ID NO:2; or (iii) SEQ ID NO:3 or an amino acid sequence that has at least 80% identity to at least 99% identity to SEQ ID NO:3; or (iv) SEQ ID NO:4 or an amino acid sequence that has at least 80% identity to at least 99% identity to SEQ ID NO:4, fused to (b) a C-terminal region comprising an amino acid sequence that corresponds to amino acid 339, 340, 341, 342, 343, 344, 345, 346, 347, 348, 349, 350, 351, 352 or 353 to amino acid 488, 489 or 490 of (i) SEQ ID NO:1, or an amino acid sequence that has at least 80% identity to at least 99% identity to SEQ ID NO:1; or (ii) SEQ ID NO:2 or an amino acid sequence that has at least 80% identity to at least 99% identity to SEQ ID NO:2; or (iii) SEQ ID NO:3 or an amino acid sequence that has at least 80% identity to at least 99% identity to SEQ ID NO:3; or c) an N-terminal region comprising an amino acid sequence that corresponds to amino acid 1 to about amino acid 363 of SEQ ID NO:17, fused to (i) a C-terminal region comprising an amino acid sequence that corresponds to about amino acid 347 to about amino acid 489 of SEQ ID NO:1 or an amino acid sequence having at least 80% identity to at least 99% identity to SEQ ID NO:1; or (ii) a C-terminal region comprising an amino acid sequence that corresponds to about amino acid 346 to about amino acid 488 of SEQ ID NO:2, or an amino acid sequence having at least 80% identity to at least 99% identity to SEQ ID NO:2.

51.-53. (canceled)


SEQUENCE LISTING (23 sequences)

SEQ ID NO: 1 - protein, 489 aa, Serratia proteamaculans
MKIESSKVEGLFSSSFTRVNAVPLPSDTLPGVGIIGCGYNPFLAYADASAVLHPILDWSKSQFNEITMNGQQYQLPDVLQAVWLSNQSYASVTGKSLQSYLTELANSIKVSGNYGFFSASATNEFSDSSLRKSENEFSRCQQSFDLWSISIPADIARLQNYVSDDFIKLINAINPESKDSIATVFNVYGSHVLMSGVMGGKAHVSASANKLTLTQKFEMSTIVQAKYEQLTSQLSVEDKLKYSEAFDSFSESGSYTYDILGGSPSLGALVFKNNSQGSSDDNLKNWIQSISSMPVLTKFIDQTSLMPVWLLCEDKTKADALKKYYDNTWSKSQMAVASLRANYIDELTFVLGDNSDIPAPVGYTKVPIDLNSDAGGKYVYLCYHEAQFTPVNGKQPIVDIQVLYGSQMPAPGYIKIDVDLNSGAGGEFVYLSYKKGEPTSSDVINKITAVYGKNEYVPTPYGYKQISGDLNAGAGGDFVYLCTYQGGTE

SEQ ID NO: 2 - protein, 488 aa, Serratia plymuthica
MKFESLKVENLFSSSLTRINAIPLPNDALPGVGIIGCGYNPFLAYADSSAVLHPVLDWSKSQFHTVTMNGQTYQLPEILNAVWLSNQTYSSVSGKSLQSYLTELSNSIKVSGNYGFFSASATNEFTDSSLRKSENEFSRCQQSFDLWSISAPADIARLQNYISDDFKKLISSIDPNNQQTLATIFNVYGSHILMSGVMGGKAHVSASANKLTLTQKFEMSTIVQAKYEQLTSQLSAEDKLKYSEAFESFSESGSYTYDILGGSPSLGALVFKVNNQDSDDNLRKWIDSISTMPVLTKFIDQTSLLPVWTLCEDQVKADALKNYYNNTWSQLQMAVARLRANYIDEVTFVLGDNSDIPAPAGYTKVPVDLNSGAGGKFIYLCYHEAQFTPVNSKQAIVGLQVLYGKQEPAPDYSRINIDLNSGANGDDVYLSYKKGDATSKEVINKITAVYGKDQYVPTPYGYKQIPGDLNSGAGGDFVYFCTYQGGTE

SEQ ID NO: 3 - protein, 489 aa, Serratia quinivorans
MKIESSKVEGLFSSSFTRVNAVPLPSDTLPGVGIIGCGYNPFLAYADASAVLHPILDWSKSQFHEITMNGQQYQLPDVLQAVWLSNQSYASVTGKSLQSYLTELANSIKVSGNYGFFSASATNEFSDSSLRKSENEFSRCQQSFDLWSISIPADIARLQNYVSDDFIKLINAINPESKDSLATVFNVYGSHVLMSGVMGGKAHVSASANKLTLTQKFEMSTIVQAKYEQLTSQLSVEDKLKYSEAFDSFSESGSYTYDILGGSPSLGALVFKANSQGSSDDNLKSWIQSISSMPVLTKFIDQTSLMPVWLLCEDKTKADALKKYYDNTWSKSQMAVASLRANYIDELTFVLGDNSDIPAPVGYTKVPIDLNSDAGGKYVYLCYHEAQFTPVNGKQPIVDIQVLYGSQMPAPGYIKIDVDLNSGAGGEFVYLSYKKGEPTSSDVINKITAVYGKNEYVPTPYGYKQISGDLNAGAGGDFVYLCTYQGGTE

SEQ ID NO: 4 - protein, 490 aa, Woodsholea maritima
MKSLDHVAHQNLLNEPTHHKSNTKAALMRHQENLVERYLPGVEVIGAGYNPFGVYASTDSVTVQLFDWQSAPSEPVIFNPDYIAPKAVSVQQNDEARYTNVSGKTINTFQKNFSLKVTVAGSYNLFSGSVSNEFSSSETRNAENEFSRIQQSIRVWSLRLAYTDSLREYLKADVRDYIDSIQSDAQIEILFDRYGSHFLTGVVMGGAAIMASSTNKVQVDHTYENETIAKASYEALTGQISAETAAKYRQSMSSFSQNSDIHKIVVGGDGVAGAKVYSGDKADFDAWADTVGTSPDFVDFVSSVPMLGIWELCKDDAQAKKMEDYYNNTWAPRKSKEAQIYADYIDAVEVIQSNSSGVRPPSGYTKIDYDLNKGAGGDYIYLCYHKARYSAYSENKDCVSDLIIIKGNGARAPSGYTKIDVDLNEDAGGKYLYLCYKKQSYDNVEAIKGLAVVGGDNSHTPAPYGYRRIDTDVNEGAGGEYIYICYSKGA

SEQ ID NO: 5 - protein, 489 aa, Artificial Sequence
  Spro-Sply hybrid insecticidal protein.
MKIESSKVEGLFSSSFTRVNAVPLPSDTLPGVGIIGCGYNPFLAYADASAVLHPILDWSKSQFNEITMNGQQYQLPDVLQAVWLSNQSYASVTGKSLQSYLTELANSIKVSGNYGFFSASATNEFSDSSLRKSENEFSRCQQSFDLWSISIPADIARLQNYVSDDFIKLINAINPESKDSIATVFNVYGSHVLMSGVMGGKAHVSASANKLTLTQKFEMSTIVQAKYEQLTSQLSVEDKLKYSEAFDSFSESGSYTYDILGGSPSLGALVFKNNSQGSSDDNLKNWIQSISSMPVLTKFIDQTSLMPVWLLCEDKTKADALKKYYDNTWSKSQMAVASLRANYIDEVTFVLGDNSDIPAPAGYTKVPVDLNSGAGGKFIYLCYHEAQFTPVNSKQAIVGLQVLYGKQEPAPDYSRINIDLNSGANGDDVYLSYKKGDATSKEVINKITAVYGKDQYVPTPYGYKQIPGDLNSGAGGDFVYFCTYQGGTE

SEQ ID NO: 6 - protein, 488 aa, Artificial Sequence
  Sply-Spro hybrid insecticidal protein.
MKFESLKVENLFSSSLTRINAIPLPNDALPGVGIIGCGYNPFLAYADSSAVLHPVLDWSKSQFHTVTMNGQTYQLPEILNAVWLSNQTYSSVSGKSLQSYLTELSNSIKVSGNYGFFSASATNEFTDSSLRKSENEFSRCQQSFDLWSISAPADIARLQNYISDDFKKLISSIDPNNQQTLATIFNVYGSHILMSGVMGGKAHVSASANKLTLTQKFEMSTIVQAKYEQLTSQLSAEDKLKYSEAFESFSESGSYTYDILGGSPSLGALVFKVNNQDSDDNLRKWIDSISTMPVLTKFIDQTSLLPVWTLCEDQVKADALKNYYNNTWSQLQMAVARLRANYIDELTFVLGDNSDIPAPVGYTKVPIDLNSDAGGKYVYLCYHEAQFTPVNGKQPIVDIQVLYGSQMPAPGYIKIDVDLNSGAGGEFVYLSYKKGEPTSSDVINKITAVYGKNEYVPTPYGYKQISGDLNAGAGGDFVYLCTYQGGTE

SEQ ID NO: 7 - protein, 489 aa, Artificial Sequence
  SproCRW-WoodsCRW hybrid protein.
MKIESSKVEGLFSSSFTRVNAVPLPSDTLPGVGIIGCGYNPFLAYADASAVLHPILDWSKSQFNEITMNGQQYQLPDVLQAVWLSNQSYASVTGKSLQSYLTELANSIKVSGNYGFFSASATNEFSDSSLRKSENEFSRCQQSFDLWSISIPADIARLQNYVSDDFIKLINAINPESKDSIATVFNVYGSHVLMSGVMGGKAHVSASANKLTLTQKFEMSTIVQAKYEQLTSQLSVEDKLKYSEAFDSFSESGSYTYDILGGSPSLGALVFKNNSQGSSDDNLKNWIQSISSMPVLTKFIDQTSLMPVWLLCEDKTKADALKKYYDNTWSKSQMAVASLRANYIDAVEVIQSNSSGVRPPSGYTKIDYDLNKGAGGDYIYLCYHKARYSAYSENKDCVSDLIIIKGNGARAPSGYTKIDVDLNEDAGGKYLYLCYKKQSYDNVEAIKGLAVVGGDNSHTPAPYGYRRIDTDVNEGAGGEYIYICYSKGA

SEQ ID NO: 8 - protein, 490 aa, Artificial Sequence
  WoodsCRW-SproCRW hybrid protein.
MKSLDHVAHQNLLNEPTHHKSNTKAALMRHQENLVERYLPGVEVIGAGYNPFGVYASTDSVTVQLFDWQSAPSEPVIFNPDYIAPKAVSVQQNDEARYTNVSGKTINTFQKNFSLKVTVAGSYNLFSGSVSNEFSSSETRNAENEFSRIQQSIRVWSLRLAYTDSLREYLKADVRDYIDSIQSDAQIEILFDRYGSHFLTGVVMGGAAIMASSTNKVQVDHTYENETIAKASYEALTGQISAETAAKYRQSMSSFSQNSDIHKIVVGGDGVAGAKVYSGDKADFDAWADTVGTSPDFVDFVSSVPMLGIWELCKDDAQAKKMEDYYNNTWAPRKSKEAQIYADYIDELTFVLGDNSDIPAPVGYTKVPIDLNSDAGGKYVYLCYHEAQFTPVNGKQPIVDIQVLYGSQMPAPGYIKIDVDLNSGAGGEFVYLSYKKGEPTSSDVINKITAVYGKNEYVPTPYGYKQISGDLNAGAGGDFVYLCTYQGGTE

SEQ ID NO: 9 - protein, 488 aa, Artificial Sequence
  SplyCRW-WoodsCRW hybrid protein.
MKFESLKVENLFSSSLTRINAIPLPNDALPGVGIIGCGYNPFLAYADSSAVLHPVLDWSKSQFHTVTMNGQTYQLPEILNAVWLSNQTYSSVSGKSLQSYLTELSNSIKVSGNYGFFSASATNEFTDSSLRKSENEFSRCQQSFDLWSISAPADIARLQNYISDDFKKLISSIDPNNQQTLATIFNVYGSHILMSGVMGGKAHVSASANKLTLTQKFEMSTIVQAKYEQLTSQLSAEDKLKYSEAFESFSESGSYTYDILGGSPSLGALVFKVNNQDSDDNLRKWIDSISTMPVLTKFIDQTSLLPVWTLCEDQVKADALKNYYNNTWSQLQMAVARLRANYIDAVEVIQSNSSGVRPPSGYTKIDYDLNKGAGGDYIYLCYHKARYSAYSENKDCVSDLIIIKGNGARAPSGYTKIDVDLNEDAGGKYLYLCYKKQSYDNVEAIKGLAVVGGDNSHTPAPYGYRRIDTDVNEGAGGEYIYICYSKGA

SEQ ID NO: 10 - protein, 490 aa, Artificial Sequence
  WoodsCRW-SplyCRW hybrid protein.
MKSLDHVAHQNLLNEPTHHKSNTKAALMRHQENLVERYLPGVEVIGAGYNPFGVYASTDSVTVQLFDWQSAPSEPVIFNPDYIAPKAVSVQQNDEARYTNVSGKTINTFQKNFSLKVTVAGSYNLFSGSVSNEFSSSETRNAENEFSRIQQSIRVWSLRLAYTDSLREYLKADVRDYIDSIQSDAQIEILFDRYGSHFLTGVVMGGAAIMASSTNKVQVDHTYENETIAKASYEALTGQISAETAAKYRQSMSSFSQNSDIHKIVVGGDGVAGAKVYSGDKADFDAWADTVGTSPDFVDFVSSVPMLGIWELCKDDAQAKKMEDYYNNTWAPRKSKEAQIYADYIDEVTFVLGDNSDIPAPAGYTKVPVDLNSGAGGKFIYLCYHEAQFTPVNSKQAIVGLQVLYGKQEPAPDYSRINIDLNSGANGDDVYLSYKKGDATSKEVINKITAVYGKDQYVPTPYGYKQIPGDLNSGAGGDFVYFCTYQGGTE

SEQ ID NO: 11 - DNA, 1467 bp, Artificial Sequence
  SplyNtSproCt E. coli optimized sequence
atgaagttcgaaagccttaaggtggagaacctgttttcgagttcactgacccggatcaatgccattccccttcccaatgacgcattacctggtgttgggattattgggtgtggatacaacccttttctggcctatgcggactcatctgctgtcttacatcctgtgttggactggtcaaagagtcaatttcatacggttactatgaacggacaaacatatcaattgccggaaatccttaatgccgtgtggttaagtaaccaaacttactcatcggtctctggaaaaagtctgcaaagttatctgaccgagctttctaattcaatcaaagttagtggaaattatggatttttttccgcgtctgccacgaacgaattcaccgattcatccttaagaaaatccgaaaatgaattcagccgttgtcagcagtctttcgacttatggtctataagcgcccctgcagatatagcacgcctgcagaattatataagtgacgattttaagaagcttatttcttcgatagatcccaacaaccagcaaacattggctacaatattcaacgtgtatggatctcatatactgatgagcggcgtcatgggcggaaaagcccacgtctcggcgagtgcgaataagcttaccttaactcagaaatttgagatgtccacgattgttcaggcaaaatacgagcagcttacatcacaactgtcggccgaagacaagttaaagtacagcgaggcgttcgaatcattctccgagtccggttcctacacctatgatatcttaggaggttcacccagtctgggagcattggtcttcaaggttaacaaccaggattctgacgataatttacggaagtggatcgattcgatctccaccatgcctgtgttgacgaaatttatcgatcagaccagtttattgcctgtctggacgctttgcgaagatcaggtgaaagccgacgccctgaagaactactataacaacacatggtcgcagttgcagatggccgttgctagattacgggccaactacatcgatgaactgaccttcgtcctcggtgataattctgacattcctgcgccggtaggttatacgaaagtcccgatcgatcttaattcggatgccggcggtaaatacgtttatctgtgctatcacgaagcccagtttaccccggtgaacggcaaacagcccatcgtggatattcaggttctgtacggttctcagatgcctgcgccggggtatattaagattgatgtagatctgaacagcggggccggtggtgaatttgtgtacctgtcctataaaaaaggggagccgacatcgtccgatgttattaacaaaatcaccgcggtttatggaaagaatgaatatgtaccgaccccgtatggctataaacagatcagcggcgacctcaacgctggggcggggggtgacttcgtttatttgtgtacttatcaaggggggaccgaataa

SEQ ID NO: 12 - DNA, 1470 bp, Artificial Sequence
  SproNtSplyCt E. coli optimized sequence
atgaaaattgagagcagcaaggtcgaaggccttttctcctctagcttcacacgggtgaatgctgtgccgcttccgagtgacaccctgccgggtgtaggtatcattggttgcggttataatccgtttctggcgtatgccgacgctagcgccgttctgcacccgattctcgattggtcaaagtcacaatttaatgaaattaccatgaatggccagcagtaccaacttcctgatgtccttcaagcagtgtggttaagcaaccagtcgtacgcctctgtgacaggcaaatcgctccagtcataccttactgaactcgcgaactctatcaaagtctctggcaattatggatttttttcggcctctgccacgaacgaatttagcgactcgtcactgcgcaaatcagaaaacgaattttcacgctgccagcaaagttttgatttatggtctatttcgatccctgctgatattgcccgcctccagaactacgtgtcggatgactttattaaactcatcaatgcaattaatccagagtccaaagattctatcgccaccgtgtttaacgtctacgggtcccatgttttaatgtctggtgtaatgggggggaaagcgcacgttagcgcctcggctaataagcttaccctgacccagaaatttgaaatgtccaccattgtacaagctaaatacgaacagcttaccagtcagctgagcgtagaagacaagctgaaatatagtgaagcgttcgattcgttttcggagtctggctcgtatacctacgatatcctgggcggttcgccgtccttaggggctttagttttcaaaaacaatagtcagggctcgtctgacgataaccttaagaactggatccaatctattagctcgatgccagtacttaccaaattcatcgaccagacaagcctgatgcccgtttggctgttgtgcgaggataaaaccaaagccgatgcgctgaaaaaatattacgataacacctggtccaagagccaaatggccgtcgcaagcctgcgcgcgaactacattgacgaggtaacgtttgttcttggcgataactctgacatccctgcgccagcagggtatactaaggtaccagtggacctgaattctggggcaggtggtaaattcatttatctttgctatcatgaagcgcagtttactcccgtgaactctaagcaagccatagttggcttgcaggtcttgtacgggaaacaagaaccagcacctgattattcacgtattaacatagatctgaattctggagcaaacggggatgatgtctacctttcttataaaaagggggacgccacttcgaaagaagtcatcaacaaaatcactgcggtgtacgggaaggatcagtacgttcccacaccgtatggctacaagcaaattccgggggacctgaattcaggtgcgggtggcgactttgtctatttttgtacttatcagggaggaactgaataa

SEQ ID NO: 13 - DNA, 1470 bp, Artificial Sequence
  SproNt-WoodsCt E. coli optimized sequence
atgaaaattgagagcagcaaggtcgaaggccttttctcctctagcttcacacgggtgaatgctgtgccgcttccgagtgacaccctgccgggtgtaggtatcattggttgcggttataatccgtttctggcgtatgccgacgctagcgccgttctgcacccgattctcgattggtcaaagtcacaatttaatgaaattaccatgaatggccagcagtaccaacttcctgatgtccttcaagcagtgtggttaagcaaccagtcgtacgcctctgtgacaggcaaatcgctccagtcataccttactgaactcgcgaactctatcaaagtctctggcaattatggatttttttcggcctctgccacgaacgaatttagcgactcgtcactgcgcaaatcagaaaacgaattttcacgctgccagcaaagttttgatttatggtctatttcgatccctgctgatattgcccgcctccagaactacgtgtcggatgactttattaaactcatcaatgcaattaatccagagtccaaagattctatcgccaccgtgtttaacgtctacgggtcccatgttttaatgtctggtgtaatgggggggaaagcgcacgttagcgcctcggctaataagcttaccctgacccagaaatttgaaatgtccaccattgtacaagctaaatacgaacagcttaccagtcagctgagcgtagaagacaagctgaaatatagtgaagcgttcgattcgttttcggagtctggctcgtatacctacgatatcctgggcggttcgccgtccttaggggctttagttttcaaaaacaatagtcagggctcgtctgacgataaccttaagaactggatccaatctattagctcgatgccagtacttaccaaattcatcgaccagacaagcctgatgcccgtttggctgttgtgcgaggataaaaccaaagccgatgcgctgaaaaaatattacgataacacctggtccaagagccaaatggccgtcgcaagcctgcgcgcgaactacatcgatgcagtcgaagtgatccagagtaattcttcgggggttcgccctcccagtggatatacgaaaattgattacgacctcaataaaggcgccggtggggattatatctacctgtgttatcataaagcccgttatagcgcttactcagaaaacaaagattgcgtttctgatttaattattattaagggaaacggagcacgcgccccgagcggttatacgaaaattgatgtggatttgaatgaggatgcaggtggaaaatacctgtatttatgctacaagaagcaaagttacgacaatgtggaagcaatcaaggggctggcggtagttggtggtgacaattcgcatacccctgctccgtacggttaccgtcggattgacaccgatgttaacgagggggccggaggtgagtacatttatatctgttactccaaaggggcttaa

SEQ ID NO: 14 - DNA, 1473 bp, Artificial Sequence
  WoodsNt-SproCt E. coli optimized sequence
atgaaaagcttggatcacgttgctcatcagaacttactgaatgaaccgactcaccataaaagcaataccaaggcggcgctgatgcgtcatcaggaaaacctggttgaacgctatctgccgggtgtggaggtgattggggcaggttacaaccctttcggcgtatacgcgtccactgactcagttactgtgcaactttttgattggcaaagcgcgccatcagaaccggtcatttttaaccccgattacattgctccgaaagctgtgtcagtacaacaaaatgatgaagcccggtatacgaacgttagcggcaaaacaatcaacacgtttcaaaaaaacttctccctgaaagttactgtcgcgggatcctataacttatttagcggtagcgtcagcaatgaattcagcagcagcgaaactcgcaatgcagaaaacgaatttagccgcatccaacagagcatccgcgtctggtctctgcgcctcgcgtacacggattccctgcgtgaatatttaaaagcggacgttcgcgattatatcgatagcattcaatcagatgcgcagattgaaattctgtttgatcgttacgggtcacatttccttaccggcgtggtgatgggtggcgcagcaatcatggcgtcttcgaccaataaagtacaggtggaccacacatacgagaatgaaaccattgccaaggccagctacgaagccttaactgggcagatctctgctgagactgcggctaaatatcgccaaagcatgtcgtcgttctcccagaactccgatattcacaaaattgtggtcggcggagacggagtcgcgggcgcgaaagtgtatagcggtgacaaggcggatttcgatgcttgggctgacaccgtggggacgtctccagattttgtggactttgttagcagcgttcctatgttgggaatttgggaactgtgcaaagatgacgcgcaggcgaaaaagatggaagattactataataatacgtgggccccgcggaaaagcaaagaggctcaaatttatgctgattacatcgatgaactgaccttcgtcctcggtgataattctgacattcctgcgccggtaggttatacgaaagtcccgatcgatcttaattcggatgccggcggtaaatacgtttatctgtgctatcacgaagcccagtttaccccggtgaacggcaaacagcccatcgtggatattcaggttctgtacggttctcagatgcctgcgccggggtatattaagattgatgtagatctgaacagcggggccggtggtgaatttgtgtacctgtcctataaaaaaggggagccgacatcgtccgatgttattaacaaaatcaccgctgtttatggaaagaatgaatatgtaccgaccccgtatggctataaacagatcagcggcgacctcaacgctggggcggggggtgacttcgtttatttgtgtacttatcaaggggggaccgaataa

SEQ ID NO: 15 - DNA, 1467 bp, Artificial Sequence
  SplyNt-WoodsCt E. coli optimized sequence
atgaagttcgaaagccttaaggtggagaacctgttttcgagttcactgacccggatcaatgccattccccttcccaatgacgcattacctggtgttgggattattgggtgtggatacaacccttttctggcctatgcggactcatctgctgtcttacatcctgtgttggactggtcaaagagtcaatttcatacggttactatgaacggacaaacatatcaattgccggaaatccttaatgccgtgtggttaagtaaccaaacttactcatcggtctctggaaaaagtctgcaaagttatctgaccgagctttctaattcaatcaaagttagtggaaattatggatttttttccgcgtctgccacgaacgaattcaccgattcatccttaagaaaatccgaaaatgaattcagccgttgtcagcagtctttcgacttatggtctataagcgcccctgcagatatagcacgcctgcagaattatataagtgacgattttaagaagcttatttcttcgatagatcccaacaaccagcaaacattggctacaatattcaacgtgtatggatctcatatactgatgagcggcgtcatgggcggaaaagcccacgtctcggcgagtgcgaataagcttaccttaactcagaaatttgagatgtccacgattgttcaggcaaaatacgagcagcttacatcacaactgtcggccgaagacaagttaaagtacagcgaggcgttcgaatcattctccgagtccggttcctacacctatgatatcttaggaggttcacccagtctgggagcattggtcttcaaggttaacaaccaggattctgacgataatttacggaagtggatcgattcgatctccaccatgcctgtgttgacgaaatttatcgatcagaccagtttattgcctgtctggacgctttgcgaagatcaggtgaaagccgacgccctgaagaactactataacaacacatggtcgcagttgcagatggccgttgctagattacgggccaactacattgacgcagtcgaagtgatccagagtaattcttcgggggttcgccctcccagtggatatacgaaaattgattacgacctcaataaaggcgccggtggggattatatctacctgtgttatcataaagcccgttatagcgcttactcagaaaacaaagattgcgtttctgatttaattattattaagggaaacggagcacgcgccccgagcggttatacgaaaattgatgtggatttgaatgaggatgcaggtggaaaatacctgtatttatgctacaagaagcaaagttacgacaatgtggaagcaatcaaggggctggcggtagttggtggtgacaattcgcatacccctgctccgtacggttaccgtcggattgacaccgatgttaacgagggggccggaggtgagtacatttatatctgttactccaaaggggcttaa

SEQ ID NO: 16 - DNA, 1473 bp, Artificial Sequence
  WoodsNt-SplyCt E. coli optimized sequence
atgaaaagcttggatcacgttgctcatcagaacttactgaatgaaccgactcaccataaaagcaataccaaggcggcgctgatgcgtcatcaggaaaacctggttgaacgctatctgccgggtgtggaggtgattggggcaggttacaaccctttcggcgtatacgcgtccactgactcagttactgtgcaactttttgattggcaaagcgcgccatcagaaccggtcatttttaaccccgattacattgctccgaaagctgtgtcagtacaacaaaatgatgaagcccggtatacgaacgttagcggcaaaacaatcaacacgtttcaaaaaaacttctccctgaaagttactgtcgcgggatcctataacttatttagcggtagcgtcagcaatgaattcagcagcagcgaaactcgcaatgcagaaaacgaatttagccgcatccaacagagcatccgcgtctggtctctgcgcctcgcgtacacggattccctgcgtgaatatttaaaagcggacgttcgcgattatatcgatagcattcaatcagatgcgcagattgaaattctgtttgatcgttacgggtcacatttccttaccggcgtggtgatgggtggcgcagcaatcatggcgtcttcgaccaataaagtacaggtggaccacacatacgagaatgaaaccattgccaaggccagctacgaagccttaactgggcagatctctgctgagactgcggctaaatatcgccaaagcatgtcgtcgttctcccagaactccgatattcacaaaattgtggtcggcggagacggagtcgcgggcgcgaaagtgtatagcggtgacaaggcggatttcgatgcttgggctgacaccgtggggacgtctccagattttgtggactttgttagcagcgttcctatgttgggaatttgggaactgtgcaaagatgacgcgcaggcgaaaaagatggaagattactataataatacgtgggccccgcggaaaagcaaagaggctcaaatttatgctgattatatcgacgaggtaacgtttgttcttggcgataactctgacatccctgcgccagcagggtatactaaggtaccagtggacctgaattctggggcaggtggtaaattcatttatctttgctatcatgaagcgcagtttactcccgtgaactctaagcaagccatagttggcttgcaggtcttgtacgggaaacaagaaccagcacctgattattcacgtattaacatagatctgaattctggagcaaacggggatgatgtctacctttcttataaaaagggggacgccacttcgaaagaagtcatcaacaaaatcactgcggtgtacgggaaggatcagtacgttcccacaccgtatggctacaagcaaattccgggggacctgaattcaggtgcgggtggcgactttgtctatttttgtacttatcagggaggaactgaataa

SEQ ID NO: 17 - protein, 510 aa, Photrhabdus luminescens
MSNDKTGKSLEQENSERDVEIRDRNYFRKLSLFDDTVIAGAEMIGTSYDVFGKYCNVGSCMNSLFDERKINASEDNFKKVTILGKTLKVPYYIDCYSVGDLKYTNASGESIESYQSNISSKSRIKGNYLFFSASLKVDFDTDSLTDFENAFSRIQYTYDLYILKSSAEALKEFLKESVKTALDKADTEEDMNDLFNTWGSHFLSGVVMGGCAQYSSSTNKYTSNLTNSFDVVAAASFAGFIGLSARTGNSFMEDIKKFRSASNIKTHAIGGDLSRFDPFGGATSADQPSAEEIAAAKKAFEDWKASVPNAPELVNFADSNPLTGIWELCSDRTQKAKLKKHFETVWAPAESAKRRVHADYIDEIIIGINNTNTPPEGYIGLKSTKDENLNSKGNICLFMHKAKYDPNIDNKDCITELKFITVRDKSPEGDWVKIPQDIYISPNQYLYLCYLPAKYSAEKAIKDIQLLCSSCGSSMILPYGYNDVLDERGERANATEDDNVHYLIYSAGWK

SEQ ID NO: 18 - protein, 506 aa, Artificial Sequence
  Chimeric Plu1415/SproCRW.
MSNDKTGKSLEQENSERDVEIRDRNYFRKLSLFDDTVIAGAEMIGTSYDVFGKYCNVGSCMNSLFDERKINASEDNFKKVTILGKTLKVPYYIDCYSVGDLKYTNASGESIESYQSNISSKSRIKGNYLFFSASLKVDFDTDSLTDFENAFSRIQYTYDLYILKSSAEALKEFLKESVKTALDKADTEEDMNDLFNTWGSHFLSGVVMGGCAQYSSSTNKYTSNLTNSFDVVAAASFAGFIGLSARTGNSFMEDIKKFRSASNIKTHAIGGDLSRFDPFGGATSADQPSAEEIAAAKKAFEDWKASVPNAPELVNFADSNPLTGIWELCSDRTQKAKLKKHFETVWAPAESAKRRVHADYIDELTFVLGDNSDIPAPVGYTKVPIDLNSDAGGKYVYLCYHEAQFTPVNGKQPIVDIQVLYGSQMPAPGYIKIDVDLNSGAGGEFVYLSYKKGEPTSSDVINKITAVYGKNEYVPTPYGYKQISGDLNAGAGGDFVYLCTYQGGTE

SEQ ID NO: 19 - protein, 506 aa, Artificial Sequence
  Chimeric Plu1415/SplyCRW.
MSNDKTGKSLEQENSERDVEIRDRNYFRKLSLFDDTVIAGAEMIGTSYDVFGKYCNVGSCMNSLFDERKINASEDNFKKVTILGKTLKVPYYIDCYSVGDLKYTNASGESIESYQSNISSKSRIKGNYLFFSASLKVDFDTDSLTDFENAFSRIQYTYDLYILKSSAEALKEFLKESVKTALDKADTEEDMNDLFNTWGSHFLSGVVMGGCAQYSSSTNKYTSNLTNSFDVVAAASFAGFIGLSARTGNSFMEDIKKFRSASNIKTHAIGGDLSRFDPFGGATSADQPSAEEIAAAKKAFEDWKASVPNAPELVNFADSNPLTGIWELCSDRTQKAKLKKHFETVWAPAESAKRRVHADYIDEVTFVLGDNSDIPAPAGYTKVPVDLNSGAGGKFIYLCYHEAQFTPVNSKQAIVGLQVLYGKQEPAPDYSRINIDLNSGANGDDVYLSYKKGDATSKEVINKITAVYGKDQYVPTPYGYKQIPGDLNSGAGGDFVYFCTYQGGTE

SEQ ID NO: 20 - DNA, 1521 bp, Artificial Sequence
  Chimeric Plu1415/SproCRW CDS.
atgtcaaacgataaaacgggaaaaagtctggaacaggaaaatagcgagcgcgacgttgaaattcgcgatcgcaactactttcgcaaattgagcttgtttgacgatactgttatcgccggtgcggagatgatcggtactagctacgatgtgtttggcaagtactgcaatgtaggaagctgcatgaacagcctcttcgatgaacgtaaaatcaacgcgagtgaagacaactttaaaaaagttaccattttaggcaaaacgctcaaggtaccgtattatatcgattgttattctgtcggtgatcttaaatacacgaacgcatcaggcgaatctatcgagagttatcagtctaacatttcttctaaaagtcgtatcaaaggtaactatctgtttttttctgcctcactcaaggtcgatttcgatacggatagcctgacggatttcgagaatgccttctctcgtatccagtacacgtatgatttgtatattttaaaatcatccgcggaagccctcaaggaatttctgaaagagtcagtcaaaacggcgttggacaaggcggataccgaagaagacatgaacgacctgtttaatacttggggcagtcatttcctgtcaggcgtagtgatgggtggttgcgcgcaatacagttcgagtacgaataagtatacctcaaatctgaccaatagctttgacgttgttgcagcggcatctttcgctggttttattggtctgtccgcccgtacaggcaactcatttatggaagatattaaaaaatttcgctccgcatctaatattaagacccacgcgatcggtggggatctcagccgcttcgatccgttcggcggcgccacctccgccgatcaaccgtctgccgaagagatcgcggcggccaagaaggcctttgaagattggaaagccagcgtaccaaacgcaccggaattagtgaacttcgcggattctaatccgctgaccggcatttgggagctgtgctcggatcgtacgcagaaagcgaaactgaaaaaacacttcgagacggtgtgggccccggcagaaagcgcgaagcgtcgggtacatgcggattatatcgacgaactgaccttcgtcctcggtgataattctgacattcctgcgccggtaggttatacgaaagtcccgatcgatcttaattcggatgccggcggtaaatacgtttatctgtgctatcacgaagcccagtttaccccggtgaacggcaaacagcccatcgtggatattcaggttctgtacggttctcagatgcctgcgccggggtatattaagattgatgtagatctgaacagcggggccggtggtgaatttgtgtacctgtcctataaaaaaggggagccgacatcgtccgatgttattaacaaaatcaccgctgtttatggaaagaatgaatatgtaccgaccccgtatggctataaacagatcagcggcgacctcaacgctggggcggggggtgacttcgtttatttgtgtacttatcaaggggggaccgaataa

SEQ ID NO: 21 - DNA, 1521 bp, Artificial Sequence
  Chimeric Plu1415/SplyCRW CDS.
atgtcaaacgataaaacgggaaaaagtctggaacaggaaaatagcgagcgcgacgttgaaattcgcgatcgcaactactttcgcaaattgagcttgtttgacgatactgttatcgccggtgcggagatgatcggtactagctacgatgtgtttggcaagtactgcaatgtaggaagctgcatgaacagcctcttcgatgaacgtaaaatcaacgcgagtgaagacaactttaaaaaagttaccattttaggcaaaacgctcaaggtaccgtattatatcgattgttattctgtcggtgatcttaaatacacgaacgcatcaggcgaatctatcgagagttatcagtctaacatttcttctaaaagtcgtatcaaaggtaactatctgtttttttctgcctcactcaaggtcgatttcgatacggatagcctgacggatttcgagaatgccttctctcgtatccagtacacgtatgatttgtatattttaaaatcatccgcggaagccctcaaggaatttctgaaagagtcagtcaaaacggcgttggacaaggcggataccgaagaagacatgaacgacctgtttaatacttggggcagtcatttcctgtcaggcgtagtgatgggtggttgcgcgcaatacagttcgagtacgaataagtatacctcaaatctgaccaatagctttgacgttgttgcagcggcatctttcgctggttttattggtctgtccgcccgtacaggcaactcatttatggaagatattaaaaaatttcgctccgcatctaatattaagacccacgcgatcggtggggatctcagccgcttcgatccgttcggcggcgccacctccgccgatcaaccgtctgccgaagagatcgcggcggccaagaaggcctttgaagattggaaagccagcgtaccaaacgcaccggaattagtgaacttcgcggattctaatccgctgaccggcatttgggagctgtgctcggatcgtacgcagaaagcgaaactgaaaaaacacttcgagacggtgtgggccccggcagaaagcgcgaagcgtcgggtacatgcggattatatcgacgaggtaacgtttgttcttggcgataactctgacatccctgcgccagcagggtatactaaggtaccagtggacctgaattctggggcaggtggtaaattcatttatctttgctatcatgaagcgcagtttactcccgtgaactctaagcaagccatagttggcttgcaggtcttgtacgggaaacaagaaccagcacctgattattcacgtattaacatagatctgaattctggagcaaacggggatgatgtctacctttcttataaaaagggggacgccacttcgaaagaagtcatcaacaaaatcactgcggtgtacgggaaggatcagtacgttcccacaccgtatggctacaagcaaattccgggggacctgaattcaggtgcgggtggcgactttgtctatttttgtacttatcagggaggaactgaataa

SEQ ID NO: 22 - protein, 529 aa, Artificial Sequence
  Chimeric Plu1415/Hmass protein.
MSNDKTGKSLEQENSERDVEIRDRNYFRKLSLFDDTVIAGAEMIGTSYDVFGKYCNVGSCMNSLFDERKINASEDNFKKVTILGKTLKVPYYIDCYSVGDLKYTNASGESIESYQSNISSKSRIKGNYLFFSASLKVDFDTDSLTDFENAFSRIQYTYDLYILKSSAEALKEFLKESVKTALDKADTEEDMNDLFNTWGSHFLSGVVMGGCAQYSSSTNKYTSNLTNSFDVVAAASFAGFIGLSARTGNSFMEDIKKFRSASNIKTHAIGGDLSRFDPFGGATSADQPSAEEIAAAKKAFEDWKASVPNAPELVNFADSNPLTGIWELCSDRTQKAKLKKHFETVWAPAESAKRRVHADYIDEIFVGYGDTPDAARADVYAQMAAYDPKAPEFIVFKDLNCSARGKYEYLGYTVTTDLKEAIRGMRGATDPNDRCSDTYNVGGCTYHRLRRDLNHGAGGHYVYLYWTKDAAAGKPLLAADVEINYSGFDHYGQPGWSRVRLINNDGDLNTNRGTGSRTYDIFVWVSKEL

SEQ ID NO: 23 - DNA, 1590 bp, Artificial Sequence
  Chimeric Plu1415/HmassCRW CDS.
atgtcaaacgataaaacgggaaaaagtctggaacaggaaaatagcgagcgcgacgttgaaattcgcgatcgcaactactttcgcaaattgagcttgtttgacgatactgttatcgccggtgcggagatgatcggtactagctacgatgtgtttggcaagtactgcaatgtaggaagctgcatgaacagcctcttcgatgaacgtaaaatcaacgcgagtgaagacaactttaaaaaagttaccattttaggcaaaacgctcaaggtaccgtattatatcgattgttattctgtcggtgatcttaaatacacgaacgcatcaggcgaatctatcgagagttatcagtctaacatttcttctaaaagtcgtatcaaaggtaactatctgtttttttctgcctcactcaaggtcgatttcgatacggatagcctgacggatttcgagaatgccttctctcgtatccagtacacgtatgatttgtatattttaaaatcatccgcggaagccctcaaggaatttctgaaagagtcagtcaaaacggcgttggacaaggcggataccgaagaagacatgaacgacctgtttaatacttggggcagtcatttcctgtcaggcgtagtgatgggtggttgcgcgcaatacagttcgagtacgaataagtatacctcaaatctgaccaatagctttgacgttgttgcagcggcatctttcgctggttttattggtctgtccgcccgtacaggcaactcatttatggaagatattaaaaaatttcgctccgcatctaatattaagacccacgcgatcggtggggatctcagccgcttcgatccgttcggcggcgccacctccgccgatcaaccgtctgccgaagagatcgcggcggccaagaaggcctttgaagattggaaagccagcgtaccaaacgcaccggaattagtgaacttcgcggattctaatccgctgaccggcatttgggagctgtgctcggatcgtacgcagaaagcgaaactgaaaaaacacttcgagacggtgtgggccccggcagaaagcgcgaagcgtcgggtacatgcggattatatcgacgagattttcgtaggctatggggatacgccggacgcggcacgcgcagacgtatatgcacagatggcggcatacgatccgaaggccccggagtttattgtttttaaagatctgaattgttcagctcgcggtaaatatgaatatctgggatacactgtgacgaccgacctcaaagaggcgatccgcggcatgcgcggcgcaaccgacccgaatgatcgctgttccgatacctataatgtgggtgggtgtacttaccaccggctgcgtcgggacctgaaccacggagcgggtgggcattacgtgtatctgtattggaccaaagacgctgcggcgggcaagccattgcttgccgcggatgtcgagattaattattcaggcttcgatcattatgggcagcccgggtggtctcgtgttcgtttgattaataatgatggggacctcaatacgaatcgtgggacgggctcccgcacctatgacattttcgtatgggtttcaaaagaattgtaa